(19) United States
(12) Patent Application Publication
(10) Pub. No.: US 2008/0241182 A1
(43) Pub. Date: Oct. 2, 2008

(54) MYCOBACTERIUM TUBERCULOSIS PERSISTANCE GENES

(76) Inventors: Sanjay K. Jain, Baltimore, MD (US); S. Moises Hernandez-Abanto, Gaithersburg, MD (US); Gyanu Lamichhane, Baltimore, MD (US); William R. Bishai, Baltimore, MD (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 12/016,465

(22) Filed: Jan. 18, 2008

Related U.S. Application Data
(60) Provisional application No. 60/885,793, filed on Jan. 19, 2007.

(51) Int. Cl. A61K 39/00; C12N 1/20; G01N 33/00; A61P 37/00

(52) U.S. Cl. 424/190.1; 435/252.3; 435/245; 424/248.1; 436/86

(57) ABSTRACT

Compositions and methods for preventing Mycobacterium infections, particularly persistent, latent Mycobactial infections, are provided. The compositions and methods utilize newly identified Mycobacterium genes (and/or corresponding gene products) that are necessary for survival of Mycobacteriae within a host.

### CROSS-REFERENCE TO RELATED APPLICATIONS

This application claims benefit of U.S. provisional patent application 60/885,793, filed Jan. 19, 2007, the complete contents of which is hereby incorporation by reference.

This invention was made using funds from grants from the National Institutes of Health having grant numbers AI36973, AI43846, AI37856, and N01 AI30036. The United States government may have certain rights in this invention.

### BACKGROUND OF THE INVENTION

1. Field of the Invention

The invention generally relates to compositions and methods for preventing mycobacterial infections, especially persistent infections. In particular, the invention provides vaccine compositions and methodology which use newly identified mycobacterial genes (and/or corresponding gene products) that are necessary for survival/persistence of Mycobacterium within a host.

2. Background of the Invention

Tuberculosis is one of the leading infectious causes of morbidity and mortality worldwide with an estimated 8.8 million new patients developing tuberculosis disease each year [1]. Humans become infected with Mycobacterium tuberculosis via the respiratory route. After successful implantation, M. tuberculosis replicates in the lungs. This initial infection may lead to disease; however, it is frequently controlled by the host immune system, leading to latent infection. Identification of M. tuberculosis genes required for survival/persistence in mammalian tissues may lead to the identification of key targets for drug and vaccine development [2].

Several defective growth phenotypes have been described by studying the in vivo growth patterns of M. tuberculosis mutants in mice [2, 3]. However, these phenotypes have not been characterized extensively in other animal models, particularly the ones which display caseous necrosis—the hallmark of the immune response to M. tuberculosis in humans. High-throughput techniques such as TraSH (Transposon Site Hybridization) have been developed previously for the subtractive identification of attenuated transposon (Tn) mutants based upon microarray analysis [4, 5]. Recently, Lamichhane et al have described Designer Arrays for Defined Mutant Analysis (DeADMAn), a high-throughput, high-sensitivity approach for subtractive identification of previously archived, genotypically-defined M. tuberculosis Himar1 Tn mutants attenuated for survival in mice after intravenous infection [6].

There is an ongoing need to provide compositions and methods for successfully preventing infection by mycobacteria, and in particular for preventing the establishment and maintenance of a persistent, latent mycobacterial infection.

### SUMMARY OF THE INVENTION

The present invention provides compositions and methods for successfully preventing infection by Mycobacterium, and in particular for preventing the establishment and maintenance of persistent, latent Mycobacterium infections. The present invention is based on the identification of Mycobacterium tuberculosis (Mtb) genes that are necessary for survival/persistence of Mtb within a host. These essential genes are required by Mtb in order for the bacterium to establish and maintain an infection, particularly in order to establish and maintain a persistent, latent infection within a host. For example, compositions containing one or more of the recombinant proteins encoded by the genes may be administered to an individual in order to elicit an immune response (humoral and/or cellular) to the proteins. Alternatively, the genes themselves may be administered directly as a DNA vaccine, in which case the proteins are translated within the host. The genes may also be incorporated in a host genome or in a plasmid in a host, where the genes are expressed by the host, and where the host (e.g. recombinant BCG) is administered for vaccination and for immune stimulation purposes. In addition, identification of these genes permits the development of attenuated Mycobacterium for use as vaccinating agents. In such attenuated Mycobacterium, the genes associated with persistence are mutated such that their gene product is either totally or partially inactive. Inactivation of the protein produces a bacterium that is non-pathogenic, and that lacks the ability to establish and maintain an infection within a host, yet which can elicit an immune response to other antigenic determinants.

It is an object of this invention to provide a composition suitable for vaccination or eliciting an immune response, comprising a. at least one recombinant protein encoded by: a gene represented by i) an MT number from the Mtb CDC 1551 genome selected from the group consisting of 0361, 1102, 2050, 2500 and 2884, or ii) an Rv number from the Mtb H37Rv genome selected from the group consisting of 0346c, 1072, 1994c, 2427c, and 2817c; or a homologous gene corresponding to i) or ii); or b. at least one nucleic acid molecule encoding said at least one recombinant protein; or c. a host harboring at least one of said nucleic acid molecules. In one embodiment, at least one of the above listed nucleic acid sequences is part of a vector, for example, a plasmid or viral vector. In another embodiment, the host includes the at least one nucleic acid molecule incorporated into its genome or as a plasmid. The composition may further comprise a physiologically compatible carrier. The host may be, for example, yeast, bacteria (e.g. Escherichia coli, BCG, other mycobacteria, etc.), or any other nucleic acid delivery or protein expression system.

The invention also provides compositions that vaccinate against persistent mycobacterial infection. The composition comprises a. at least one recombinant protein encoded by: a gene represented by i) an MT number from the Mtb CDC 1551 genome selected from the group consisting of 0361, 1102, 2050, 2500, 2884, 3097 and 3978, or ii) an Rv number from the Mtb H37Rv genome selected from the group consisting of 0346c, 1072, 1994c, 2427c, 2817c, 3017c and 3864; or a homologous gene corresponding to i) or ii); or b. at least one nucleic acid molecule encoding said at least one recombinant protein;

or c. a host harboring at least one of said nucleic acid molecules. In one embodiment, at least one nucleic acid is part of a vector such as a plasmid or viral vector. In another embodiment, the host includes the at least one nucleic acid molecule incorporated into its genome or as a plasmid. The composition may include a physiologically compatible carrier. The host may be, for example, yeast, bacteria (e.g. Escherichia coli, BCG, other mycobacteria, etc.), or any other nucleic acid delivery or protein expression system.

The invention also provides a method of vaccinating an individual against persistent mycobacterial infection. The method comprises the step of administering to said individual a vaccine composition, comprising a. at least one recombinant protein encoded by: a gene represented by i) an MT number from the Mtb CDC 1551 genome selected from the group consisting of 0361, 1102, 2050, 2500, 2884, 3097 and 3978, or ii) an Rv number from the Mtb H37Rv genome selected from the group consisting of 0346c, 1072, 1994c, 2427c, 2817c, 3017c and 3864; or a homologous gene corresponding to i) or ii); or b. at least one nucleic acid molecule encoding said at least one recombinant protein; or c. a host harboring at least one of said nucleic acid molecules. In one embodiment, at least one nucleic acid is part of a vector such as a plasmid or viral vector. In another embodiment, the host includes the at least one nucleic acid molecule incorporated into its genome or as a plasmid. The composition may include a physiologically compatible carrier. The host may be, for example, yeast, bacteria (e.g. Escherichia coli, BCG, other mycobacteria, etc.), or any other nucleic acid delivery or protein expression system. The step of administering may be carried out via various routes including but not limited to intravenous, aerosol, subcutaneous, intradermal, and oral routes.

The invention further provides an attenuated, non-pathogenic mycobacteria. The attenuated mycobacteria contains at least one mutation in at least one gene selected from the group consisting of i) an MT number from the Mtb CDC 1551 genome selected from the group consisting of 0361, 1102, 2050, 2500, 2884, 3097 and 3978; ii) an Rv number from the Mtb H37Rv genome selected from the group consisting of 0346c, 1072, 1994c, 2427c, 2817c, 3017c and 3864; and iii) a homologous gene corresponding to i) or ii); wherein said at least one mutation causes a reduction or elimination of a biological activity or function of a protein or polypeptide encoded by said at least one gene. In one embodiment, the mycobacteria is Mycobacterium tuberculosis.

The invention further provides a method of vaccinating an individual against mycobacterial infection, the method comprising the step of administering to said individual a composition comprising an attenuated, non-pathogenic mycobacteria, wherein said attenuated mycobacteria contains at least one mutation in at least one gene selected from the group consisting of i) an MT number from the Mtb CDC 1551 genome selected from the group consisting of 0361, 1102, 2050, 2500, 2884, 3097 and 3978; ii) an Rv number from the Mtb H37Rv genome selected from the group consisting of 0346c, 1072, 1994c, 2427c, 2817c, 3017c and 3864; and iii) a homologous gene corresponding to i) or ii);

wherein said at least one mutation causes a reduction or elimination of a biological activity or function of a protein or polypeptide encoded by said at least one gene. In one embodiment, the mycobacteria is Mycobacterium tuberculosis.

The invention also provides a pharmacological agent and/or a mechanism for identifying a pharmacological agent, that inhibits or prevents a biological activity or function of a protein encoded by a gene represented by i) an MT number from the Mtb CDC 1551 genome selected from the group consisting of 0361, 1102, 2050, 2500, 2884, 3097 and 3978; or ii) an Rv number from the Mtb H37Rv genome selected from the group consisting of 0346c, 1072, 1994c, 2427c, 2817c, 3017c and 3864; or iii) a homologous gene corresponding to i) or ii).

The invention further provides a method of treating a mycobacterial infection in a patient in need thereof, comprising the step of administering to said patient a pharmacological agent that inhibits or prevents a biological activity or function of a protein encoded by a gene represented by i) an MT number selected from the group consisting of 0361, 1102, 2050, 2500, 2884, 3097 and 3978; or ii) an Rv number selected from the group consisting of 0346c, 1072, 1994c, 2427c, 2817c, 3017c and 3864; or iii) a homologous gene corresponding to i) or ii). The invention also provides a method of vaccinating an individual against Mycobacterium infection. The method comprises the step of administering to said individual a vaccine composition, comprising a. at least one recombinant protein encoded by: a gene represented by i) an MT number selected from the group consisting of 0361, 1102, 2050, 2500 and 2884, or ii) an Rv number selected from the group consisting of 0346c, 1072, 1994c, 2427c, and 2817c; or a homologous gene corresponding to i) or ii); or b. at least one nucleic acid molecule encoding said at least one recombinant protein; or c. a host harboring at least one of said nucleic acid molecules In one embodiment, the at least one nucleic acid is part of a vector such as a plasmid or viral vector. In another embodiment, the host includes the at least one nucleic acid molecule incorporated into its genome or as a plasmid. The composition may include a physiologically compatible carrier. The host may be, for example, yeast, bacteria (e.g. Escherichia coli, BCG, other mycobacteria, etc.), or any other nucleic acid delivery or protein expression system. The step of administering may be carried out via various routes including but not limited to intravenous, aerosol, subcutaneous, intradermal, and oral routes.

### BRIEF DESCRIPTION OF THE DRAWINGS

FIGS. 1A and B. Results after microarray and real-time PCR analysis. A. Thirty four M. tuberculosis Tn mutants were found to be attenuated for survival in either in the guinea pig or mouse aerosol models after microarray analysis. There was a high degree of agreement between the mutants found to be attenuated for survival in the guinea pig compared to mouse lung (Kappa coefficient=0.63, agreement=0.83) and B. Eighteen M. tuberculosis Tn mutants were found to be attenuated for survival in either in the guinea pig or mouse aerosol models after real-time PCR analysis. Again, there was a high degree of agreement between the mutants found to be attenuated for survival in the guinea pig compared to mouse lung (Kappa coefficient=0.63, agreement=0.83). Note: JHU2583c-322 (positive control), JHU2675c-564 (negative control), JHU0842-1196 and JHU3833-375 were present in both pools A and B and yielded same results in both pools and both models.

FIGS. 2A and B. Kaplan Meyer survival comparison for attenuated mutants. Survival curve analysis for A. Eighteen M. tuberculosis Tn mutants attenuated either in the guinea pig or mouse aerosol models. Median survival times of Tn mutants in guinea pig is significantly shorter than the mouse model (Log Rank Test p value <0.0001) and B. Ten M. tuberculosis Tn mutants attenuated in both the guinea pig and mouse models. Median survival times of Tn mutants in guinea pig is significantly less than the mouse model (Log Rank Test p value=0.0003). These data show that the guinea pig model detects attenuation for survival earlier than the mouse aerosol model.

FIG. 3. Evaluation of the protective efficacy of vaccines

FIG. 4. Evaluation of the protective efficacy of Mtb strains deleted for persistence genes.

FIG. 5A-D. Exemplary nucleic acid and corresponding protein sequences isolated from M. tuberculosis H37Rv strain.

### DETAILED DESCRIPTION OF THE PREFERRED EMBODIMENTS OF THE INVENTION

Mouse and guinea pig models have been used to identify Mycobacterium tuberculosis mutants attenuated for in vivo survival/persistence in mammalian lungs. DeADMAn, a high-throughput subtractive competition assay for genotypically-defined M. tuberculosis mutants was used, and the survival of identical pools of M. tuberculosis mutants in guinea pig and mouse aerosol models were compared. In addition, selected mutants found to be attenuated in both aerosol models were also analyzed in the mouse hollow fiber model. M. tuberculosis mutants representing 74 genes were tested. Eighteen M. tuberculosis mutants were found to be attenuated for survival in both aerosol models, with 70% of selected mutants also attenuated in the mouse hollow fiber model. The majority of attenuated mutants in the mouse aerosol model were detected only after 90 days of infection. In contrast, the majority of mouse late-stage survival mutants were detected significantly earlier in the guinea pig, suggesting that differences in tuberculosis-induced lung pathology may account for this accelerated detection. (Unlike mice, M. tuberculosis-infected guinea pigs form caseating granulomas which may simulate human disease more closely.) There was a high degree of concordance between the genes identified by the two aerosol models (p<0.0003). Comparing species enabled a direct evaluation of the impact of caseous necrosis present in guinea pig tuberculosis but absent in mouse tuberculosis [7].

The discovery of the survival/persistence genes leads to the development of compositions for eliciting an immune response to and/or vaccinating a host against infection by Myobacteriae, and against the establishment and/or maintenance of persistent, latent infection by Mycobacteriae. With respect to the meaning of “persistent” in this context, Mycobacterium tuberculosis possesses the ability to remain quiescent for long periods, “reactivating” months to decades later to produce clinical disease. Various terms, including persistence, latency, dormancy, non-replicating persistence (NRP), etc have been used to describe this quiescent physiological state of M. tuberculosis. Latency generally refers to the clinical infection and not the bacteria themselves. It is estimated that one third of the world population (˜2 billion people) are latently infected with M. tuberculosis. Clinically, this stage of infection is silent and is only detected by the use of tuberculin skin tests or ex vivo analogs such as QuantiFERON-TB, etc. Latency can be viewed as an equilibrium that exists between the host and organism. Latent tuberculosis develops after an individual has been exposed to M. tuberculosis, the infection has been established, and an immune response develops to controls the pathogen resulting in a quiescent state. Though it is unclear whether M. tuberculosis can develop a state of true metabolic inactivity, there is great amount of pathologic, epidemiologic and clinical data that demonstrates conclusively that latency does occur clinically. In brief therefore, persistence can be defined as the physiological state in which M. tuberculosis can survive in tissues for months to decades without apparent replication, yet possessing the ability to resume growth and activate months to decades later to cause disease.

In some embodiments of the invention, the infection that is treated is an infection caused by Mycobacterium tuberculosis. However, those of skill in the art will recognize that the practice of the invention need not be limited to the treatment of infections caused by this species of mycobacteria, as the survival/persistence genes are likely to be required for the establishment and maintenance of infection by other mycobacteria and Actinomycetes as well.

In one embodiment of the invention, compositions containing one or more of the gene products (proteins, polypeptides or peptides) of the survival (persistence) genes, or antigenic fragments thereof, are provided. Such compositions may be administered in order to elicit an immune response to the gene product (e.g. a humoral and/or cell mediated immune response). In preferred embodiments, the immune response is a cellular immune response. Such an immune response may prevent or lessen disease symptoms associated with infection, and preferably will prevent the establishment and maintenance of a persistent, latent Mycobacterial infection in the vaccinated host.

Those of skill in the art will recognize that an antigenic fragment of a protein is a region (usually of contiguous primary structure) which elicits an immune response at least about half as robust, and preferably equal to or superior to, the intact protein. Typically, antigenic fragments or regions of a protein are those which are surface exposed, and may comprise e.g. external loops, turns, etc. or other secondary structures that are accessible to the immune system. Such determinants, when synthesized outside the framework of the intact protein, may naturally retain sufficient native structure to elicit an immune response, or may be designed according to methods that are well known so as to retain sufficient secondary structure to do so.

In another embodiment, vaccination is accomplished by administering nucleic acid sequences that encode the survival/persistence proteins, polypeptides or peptides, or antigenic fragments thereof. For example, DNA encoding the proteins may be administered as a DNA vaccine, in the form of a vector such as a plasmid, a viral vector (e.g. an adenoviral vector), or other vectors that are known in the art. In addition, the genes encoding the survival/persistence proteins, polypeptides or peptides, or antigenic fragments thereof, may be contained within a host such as a bacterial host, which is administered to an individual in need of vaccination. The genes may reside on an extrachromosomal element in such a host (e.g. a plasmid) or may be incorporated into the genetic material of the host. Suitable hosts include but are not limited to bacteria (e.g. mycobacteria, Escherichia coli, and others that are well known to those of skill in the art), yeast, etc. Such vectors and hosts may be utilized as vaccine components, and may function as the source of the survival/persistence proteins that elicit an immune response in the vaccinated host. Alternatively, such vectors and hosts may be used for other purposes, for example, for experimental research.

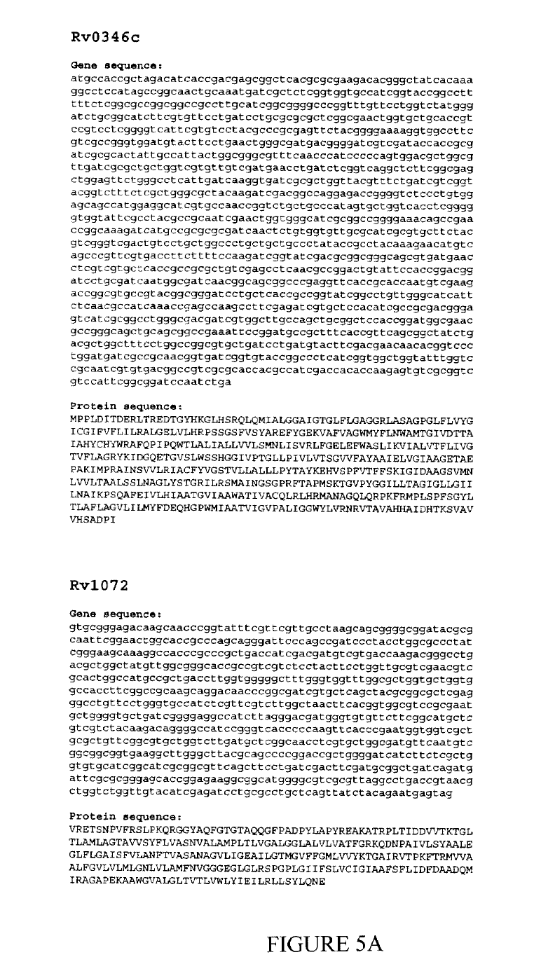

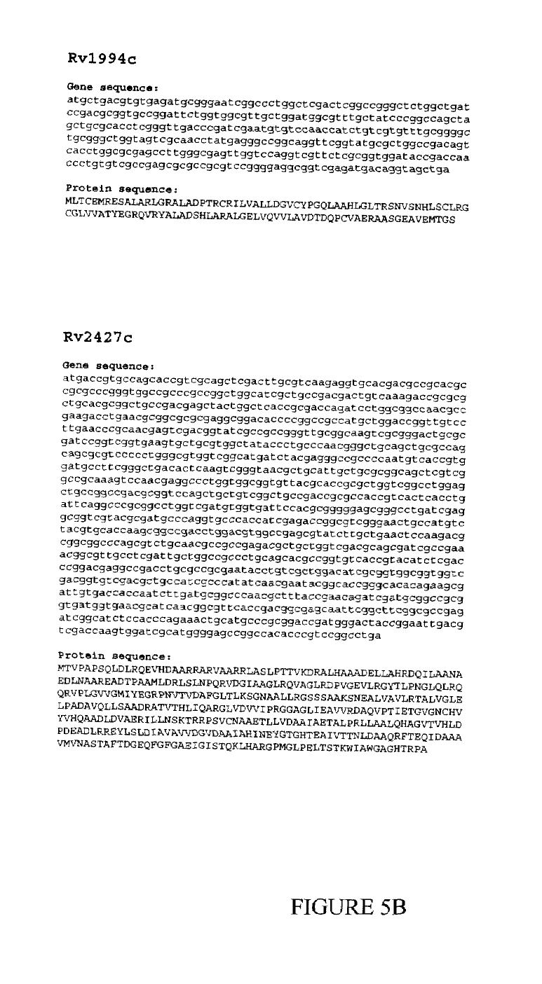

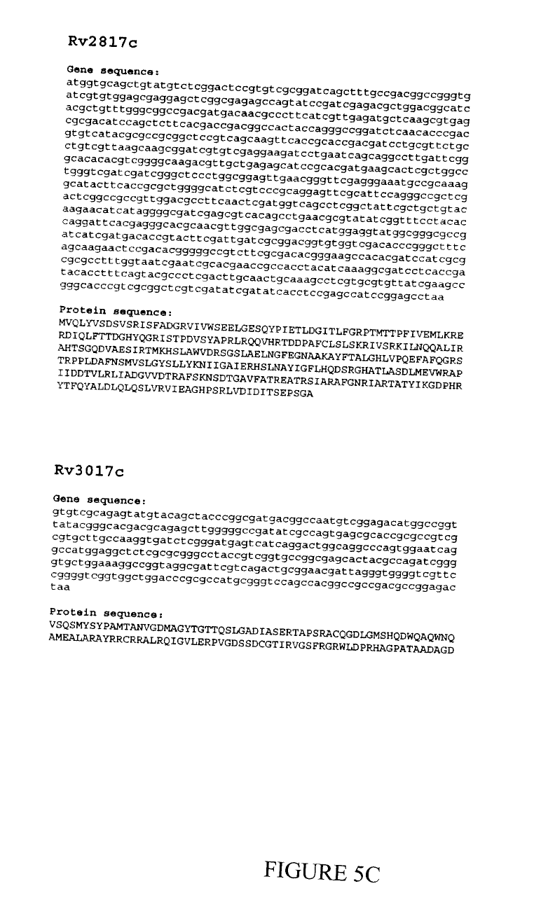

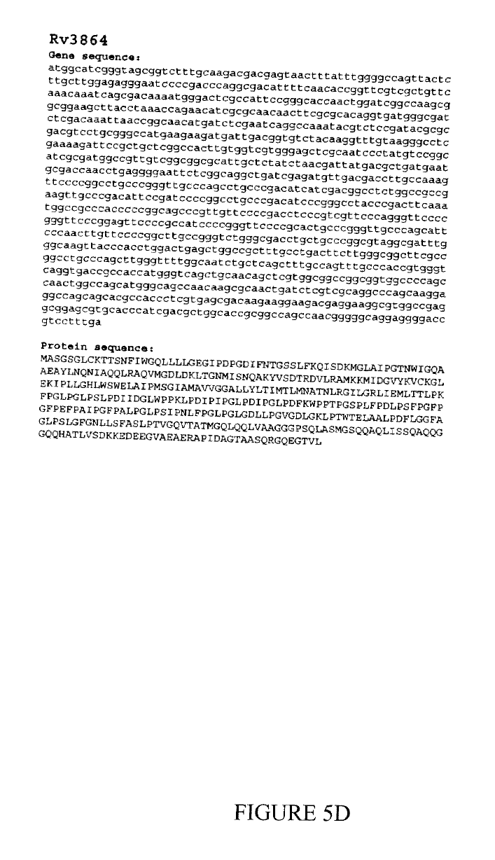

Genes that are utilized in this manner include but are not limited to genes represented by an MT number from the Mtb CDC 1551 genome selected from the group consisting of 0361, 1102, 2050, 2500, 2884, 3097 and 3978; 2) an Rv number from the Mtb H37Rv genome selected from the group consisting of 0346c, 1072, 1994c, 2427c, 2817c, 3017c and 3864; and 3) homologous genes corresponding to either 1 or 2. By “homologous genes” we mean genes that encode corresponding proteins, polypeptides or peptides that are isolated from or originate from mycobacteria other than Mtb, or from other bacteria in the Actinomycete group (e.g. Streptomyces coelicolor), and/or from strains of Mycobacterium tuberculosis other than H37RV (the source of the “Rv number”) and CDC 1551 (the source of the “MT number”). Exemplary sources also include but are not limited to bacteria classified as belonging to the Mycobacterium tuberculosis complex (M. tuberculosis, M. bovis, M. africanum, M. microti and M. canetti). In addition, those of skill in the art will recognize that the precise nucleic acid sequences that are originally identified in a mycobacterium of origin need not be utilized. Due to the redundancy of the genetic code, many alternative nucleic acid sequences may be used to encode the survival/persistence proteins identified herein. In addition, the nucleic acids that encode the survival/persistence proteins include DNA, RNA, and various hybrids thereof. Exemplary nucleic acid sequences and the corresponding gene products are depicted in FIG. 5A-D.

Those of skill in the art will recognize that the precise primary sequences of the survival/persistence proteins utilized herein also need not be retained in order to successfully practice the invention. Various changes in the primary amino acid sequence of the survival/persistence proteins may be tolerated without compromising the effectiveness of the proteins as antigens. For example, conservative (or even non-conservative) amino acid substitutions are acceptable, or deletions or additions (e.g. at the amino and/or carboxyl termini, or within the protein sequence) may be tolerated. All such variants are intended to be included in the practice of the present invention, so long as the resulting variant protein retains the ability to elicit a robust immune response in vivo, including humoral and/or cell mediated immune responses.

In addition, the recombinant proteins may be provided as chimeric or fusion proteins containing, for example, additional heterologous amino acid segments that either originate in other organisms, or are totally artificial. Examples include but are not limited to segments that are useful for protein isolation (e.g. His-tags), segments that serve to direct the protein to a particular location within the cell (e.g. leader sequences), detection tags (e.g. S-tag, or Flag-tag), other antigenic amino acid sequences such as known T-cell epitope containing sequences and protein stabilizing motifs, etc. In addition, the chimeric proteins may be chemically modified, e.g. by amidation, sulfonylation, lipidation, or other techniques that are known to those of skill in the art.

Further, more than one of the seven identified proteins may be included in a single translational product, or more than one protein may be encoded by a single nucleic acid, and still used as described herein. Alternatively, several segments containing antigenic determinants but originating from different proteins may be encoded by a single nucleic acid, and thus be included in a single translation product. In yet other embodiments, several different proteins, or several nucleic acids encoding the different proteins, are administered together in a “cocktail”. All such combinations are encompassed by the present invention.

The present invention thus provides compositions that include one or more substantially purified recombinant proteins or nucleic acids as described herein, and a pharmacologically suitable carrier. The preparation of such compositions for in vivo administration is well known to those of skill in the art. Typically, such compositions are prepared either as liquid solutions or suspensions, however solid forms such as tablets, pills, powders and the like are also contemplated. Solid forms suitable for solution in, or suspension in, liquids prior to administration may also be prepared. The preparation may also be emulsified. The active ingredients may be mixed with excipients which are pharmaceutically acceptable and compatible with the active ingredients. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. The preparations of the present invention may further comprise an adjuvant, suitable examples of which include but are not limited to Seppic, Quil A, Alhydrogel, etc. If it is desired to administer an oral form of the composition, various thickeners, flavorings, diluents, emulsifiers, dispersing aids or binders and the like may be added. The composition of the present invention may contain any such additional ingredients so as to provide the composition in a form suitable for administration. The final amount of protein or nucleic acid in the formulations may vary. However, in general, the amount in the formulations will be from about 0.01-99%, weight/volume.

The methods involve administering such a composition in a pharmacologically acceptable carrier to a mammal. The preparations of the present invention may be administered by any of the many suitable means which are well known to those of skill in the art, including but not limited to by injection, inhalation, orally, intranasally, by ingestion of a food product containing the active ingredient, etc. In preferred embodiments, the mode of administration is subcutaneous or intramuscular. In addition, the compositions may be administered in conjunction with other treatment modalities such as substances that boost the immune system, antibiotics, and the like.

The compositions of the invention may be administered to individuals at risk of being infected by mycobacteria, or to individuals who have been exposed to mycobacteria, or even to individuals known to be infected with mycobacteria. The compositions and methods of the invention may be especially useful for treating individuals who are immunocompromised (e.g. AIDS patients, patients who are receiving chemotherapy, drug addicts, etc.), or for treating persons who are known to be infected with strains of mycobacteria that are known to be drug resistant. In general, the individuals who are treated using the compositions and methods of the invention are mammals such as humans. However, the treatment of non-human mammals is in no way excluded.

The invention further provides an attenuated, non-pathogenic mycobacteria in which at least one of the survival/persistence genes or gene products is inactivated, e.g. by mutation or deletion of the gene. The inactivation is such that the bacterium does not cause disease symptoms in a host. This may be accomplished, for example, by deleting a gene, or replacing a gene with an innocuous or inactive gene, or somehow altering the gene so that the gene product is inactive or innocuous. However, the bacteria that are so-modified are useful as attenuated vaccinating agents, since they provide a rich source of antigenic determinants, without causing disease in the host. In some embodiments, only the survival/persistence genes are inactivated. However, in other embodiments, other genes may also be inactivated/deleted in addition to the survival/persistence genes, for example, the ΔsigF mutation.

The survival/persistence genes and proteins described herein may also be used to screen candidate pharmacological agents/drugs and to identify those that can be used to treat or prevent mycobacterial infections. Agents that inhibit activity of the proteins are useful for inhibiting the growth and infectivity of mycobacteria in vivo.

The survival/persistence genes and proteins described herein may also be used in various diagnostic applications, e.g. to verify the presence of mycobacteria in a host that is suspected of being infected, or in an environment that is suspected of being contaminated, and/or to identify/classify bacteria.

### EXAMPLES

### Example 1

### Accelerated Detection of Mycobacterium tuberculosis Genes Essential for Bacterial Survival in Guinea Pigs Compared with Mice

### Materials and Methods

M. tuberculosis Himar1 transposon mutants and media: Random insertion mutagenesis of M. tuberculosis CDC 1551 strain was performed in our laboratory using the Himar1 transposon (Tn) as part of a comprehensive insertional mutagenesis study. Tn insertion sites were identified by sequencing the insertion junction as described previously [8,9]. Each mutant was grown separately at 37° C. in Dubos broth base (Becton Dickinson, Sparks, Md.) supplemented with Dubos medium albumin (Becton Dickinson, Sparks, Md.), 5% glycerol, 0.01% sodium pyruvate and kanamycin at 20 μg/ml. All mutants, except for the positive and negative controls, used in this study were selected randomly. Two pools (A, B), containing 30 and 50 mutants respectively, were prepared by combining a pure culture of each mutant grown to an OD₆₀₀ of 0.8. Only mutants whose in vitro growth was similar to that of wild-type were used for the present study. Mutants that harbored a Tn in the region upstream of the terminal 100 bp (or if the gene was <500 bp in length, within the 5′ 80% of the gene) were considered for screening to decrease the likelihood of selecting distal mutations which may not disrupt gene product function. In addition, 7 M. tuberculosis Tn mutants that harbored a Tn in the terminal 100 bp (or if the gene was <500 bp in length, within the 3′ 20% of the gene) were also screened. M. tuberculosis Tn mutants JHU2583c-322 and JHU2675c-564 were included as positive and negative controls in each pool, respectively, since previous studies have shown that JHU2583c-322 (Tn mutant in gene MT2660) is attenuated for survival in mice while JHU2675c-564 (Tn mutant in gene MT2749) is fully virulent [6]. Further, M. tuberculosis Tn mutants JHU0842-1196 and JHU3833-375 (Tn mutants in genes MT0864 and MT3941, respectively) were present in both pools A and B. All mutants used in this study including the controls, were subsequently found to have a ΔsigF background [10].
Guinea pig infection: 250-300 gm Hartley strain guinea pigs (Charles River, Wilmington, Mass.) were infected via the aerosol route using the latest version of the Madison Aerosol Chamber from the University of Wisconsin Engineering Shops [11]. Five guinea pigs per group were sacrificed at days 1, 21 and 42 post-infection (pool A) and days 1, 21, 49 and 63 (pool B), and the survival of each mutant in the lungs was analyzed. Both the lungs in entirety were homogenized in phosphate-buffered saline (PBS) and a significant proportion of the entire homogenate was plated on Middlebrook 7H10 solid medium (Becton Dickinson, Sparks, Md.). For day 1 CFU counts, both the lungs in entirety were homogenized and half of the homogenate was plated as above. CFU counts were obtained by multiplying these results by a factor of two.
Mouse infection: 5-6 week-old female BALB/c mice (Charles River, Wilmington, Mass.) were infected via the aerosol route using the Glas-Col inhalation exposure system (Glas-Col, Terre Haute, Ind.). Three mice per group were sacrificed at days 1, 21, 49, 96 147 and 360 post-infection for both pools A and B, and the survival of each mutant in the lungs was analyzed. Both the lungs in entirety were homogenized in PBS and plated on Dubos broth base (Becton Dickinson, Sparks, Md.) supplemented with Dubos medium albumin (Becton Dickinson, Sparks, Md.), 1.5% agar, 5% glycerol, 0.01% sodium pyruvate and kanamycin at 20 μg/ml. For day 1 CFU counts, both the lungs in entirety were homogenized and all of the homogenate plated as above.

All plates were incubated at 37° C. for at least 3 weeks before the colonies were counted or used for DNA preparation.

Microarray and Real-Time PCR analysis: For each time point, the colonies from the plates were scraped, pooled, and genomic DNA prepared using standard methods [12].
Microarray: Two custom microarray sets (one for each pool) were printed on poly-L-lysine-coated glass slides. Sixty base oligonucleotides corresponding to the sequence at the junction of each Tn insertion were spotted four times in tandem on the microarray. The genomic DNA was digested with AluI, adapter ligated, junctions of Tn insertion sites selectively PCR amplified using transposon-specific primers, and labeled with Cy3 and Cy5 mono-fluorescent dyes (Amersham Pharmacia) as described previously [6]. Probes prepared from the input pool (1 day post-infection) and output pools were co-hybridized to custom microarrays and scanned by using Genepix Axon 4000B (Axon Instruments Inc.). Microarrays were performed at least in duplicate for each time point. The data were analyzed by using a custom developed program [6]. Mutants with the input/output pool ratio significantly greater than or equal to the average of the negative and positive controls at a given time point were considered to be attenuated.
Real-time PCR: Real-time PCR analysis was performed to confirm and quantify the microarray results for mutants found to be attenuated by DeADMAn. Mutant specific primer sets using Tn and gene specific primers were designed to amplify 150-200 bp DNA fragments. These primer sets were tested using conventional PCR. All successful primer sets were used for mutant specific real-time PCR using iCycler IQ Version 3.1.7050 (Bio-Rad, Hercules, Calif.). Cycle threshold for input pool (1 day post-infection) was compared to that for the output pool genomic DNA. For mutants not found to be attenuated at the time point indicated by microarray analysis, real-time PCR was performed at all subsequent time points available for that pool. Real-time PCR was performed at least in triplicate for each mutant.
Mouse Hollow fiber infection: Pool B culture containing 50 mutants was encapsulated into PVDF hollow fibers and implanted subcutaneously into 6-8 week old SKH1 hairless mice (Charles River, Wilmington, Mass.), as described previously [13]. Four mice (each containing two hollow fibers) were sacrificed at days 1 and 56 post-infection. The hollow fiber contents were recovered and plated on Middlebrook 7H10 solid medium (Becton Dickinson, Sparks, Md.). For each time point, the colonies from the plates were scraped, pooled, and genomic DNA prepared using standard methods [12]. Real-time PCR analysis was performed for all pool B mutants found to be attenuated for survival in the guinea pig and mouse aerosol models. Cycle threshold for input pool (1 day post-infection) was compared to that for the output pool (56 days post-infection) genomic DNA. Real-time PCR was performed at least in triplicate for each mutant.
Statistical analysis: Kaplan Meyer survival curve analysis was done using Prism 4 version 4.01 (GraphPad Software Inc., San Diego, Calif.).

### Results

M. tuberculosis Tn mutants tested and attenuated for survival in the guinea pig and mouse aerosol models: A total of 80 M. tuberculosis Tn mutants were used to infect both the guinea pig and mouse via the aerosol route (Table 1). After aerosol infection, day 1 log₁₀ CFU were 3.48±0.06 and 3.40±0.28 for pools A and B, respectively, for mouse lungs and 2.07±0.17 and 2.29±0.07 for pools A and B, respectively, for guinea pig lungs.

TABLE 1
M. tuberculosis Tn mutants tested in the guinea pig and mouse aerosol models:
Seventy six M. tuberculosis Tn mutants used for guinea pig and mouse lungs infections via the
aerosol route. Seven M. tuberculosis Tn mutants that harbor a Tn in the terminal 100 bp (or if
the gene was <500 bp in length, within the 3′ 20% of the gene) and the positive and the
negative control mutants are shaded.

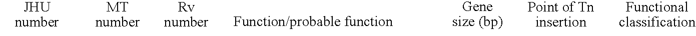

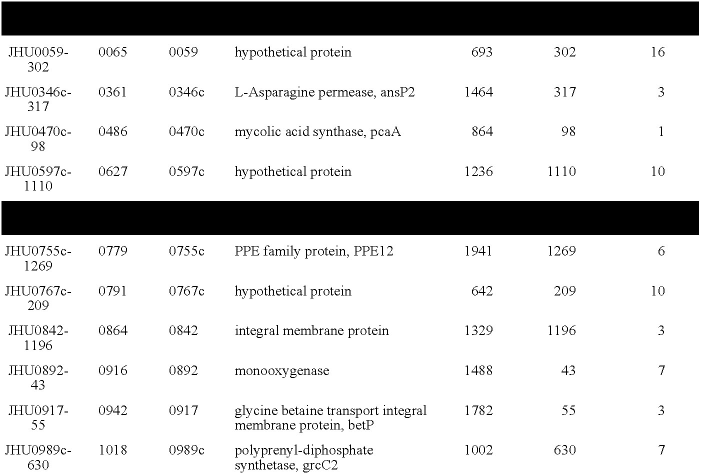

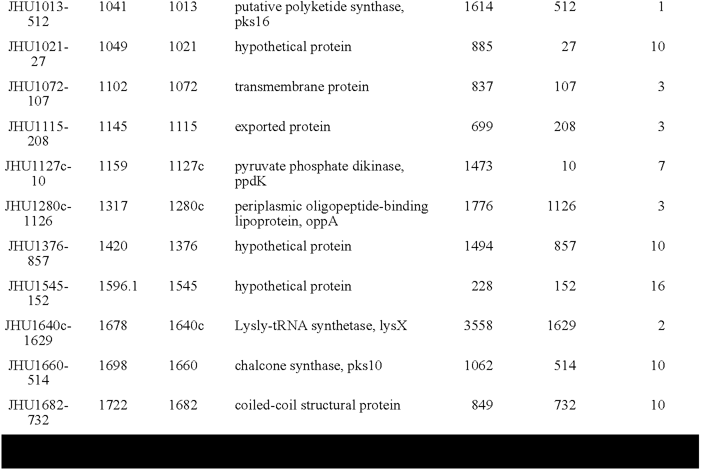

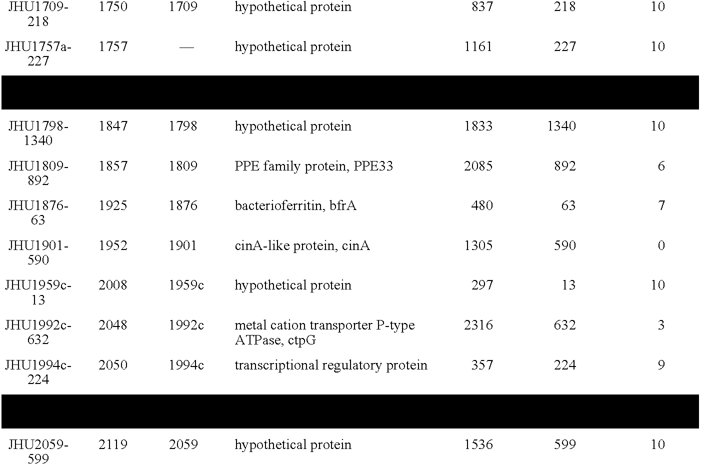

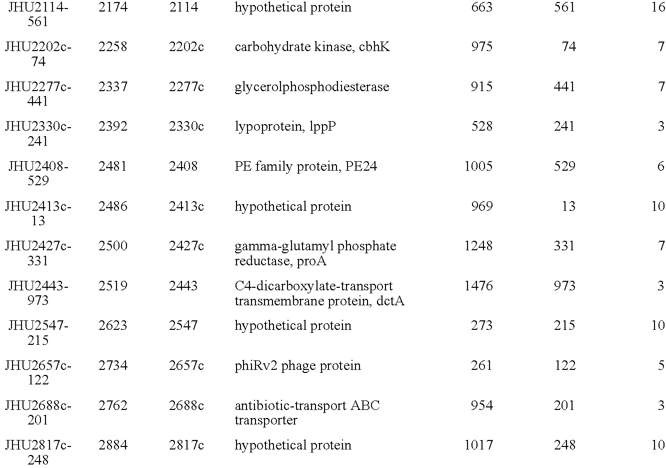

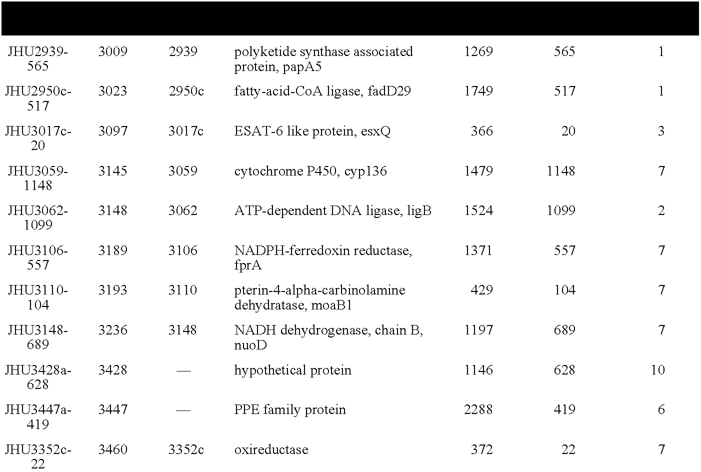

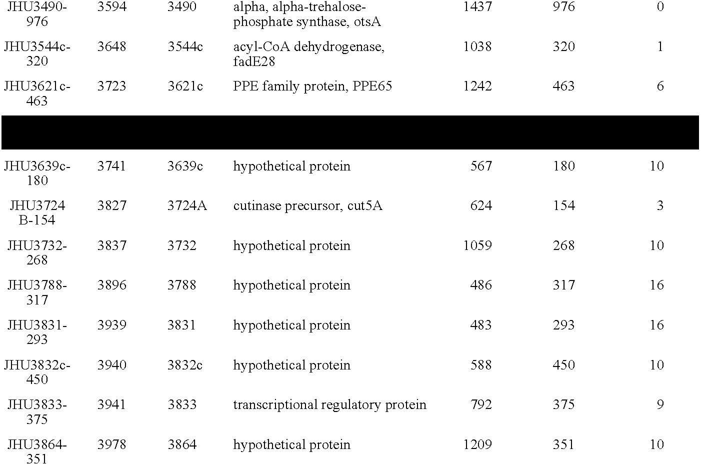

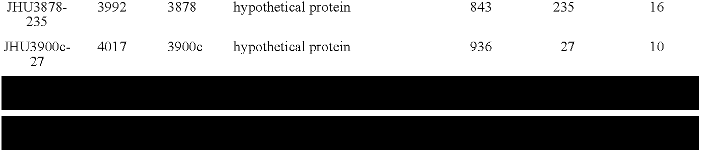

Functional classification key [14]: 0 virulence, detoxification, adaptation, 1 lipid metabolism, 2 information pathways, 3 cell wall and cell processes, 5 insertion sequences and phages, 6 PE/PPE, 7 intermediary metabolism and respiration, 8 unknown, 9 regulatory proteins, 10 conserved hypotheticals, 16 conserved hypotheticals with ortholog in M. bovis

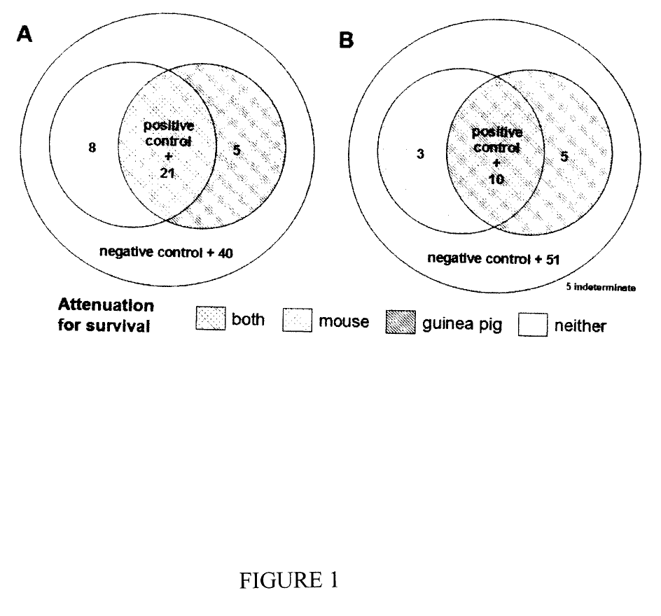

DeADMAn screening on day 1 (input pool) confirmed the lung implantation of each mutant in both pools and models used. As expected, the positive and negative controls were found to be attenuated for survival and fully virulent, respectively, in both pools and models. Similarly, M. tuberculosis Tn mutants JHU0842-1196 and JHU3833-375 were found to yield similar results in both pools and models. Thirty four M. tuberculosis Tn mutants were found to be attenuated for survival in either the guinea pig or mouse lung. Of these, 26 were found to be attenuated for survival in the guinea pig while 29 were attenuated in the mouse (FIG. 1A). There was a high degree of agreement between the mutants found to be attenuated for survival in the guinea pig compared to mouse lung (Kappa coefficient=0.63, agreement=0.83). To confirm and quantify the DeADMAn results, real-time PCR was performed for the 34 M. tuberculosis Tn mutants attenuated for survival. Primer sets for 5 mutants (JHU1021-27, JHU1127c-10, JHU2202c-74, JHU3447a-419 and JHU3352c-22) were unsuccessful in amplifying a specific PCR product and were removed from further analysis. Eighteen mutants were found to be attenuated for survival in either the guinea pig or mouse aerosol models. Of these, 15 were found to be attenuated for survival in the guinea pig while 13 were attenuated in the mouse (Table 2). Again, there was a high degree of agreement between the mutants found to be attenuated for survival in the guinea pig compared to mouse lung (Kappa coefficient=0.64, agreement=0.89) suggesting a high degree of concordance for M. tuberculosis genetic requirements for growth between the two models (FIG. 1B).

TABLE 2
M. tuberculosis Tn mutants found to be attenuated for survival in the guinea pig
and mouse aerosol models. Eighteen M. tuberculosis Tn mutants found to be attenuated for
survival in either the guinea pig or mouse aerosol models. Fifteen were found to be attenuated
for survival in the guinea pig while 13 were attenuated in the mouse aerosol model with a high
degree of concordance between the two models. Of these, 7 of 10 mutants tested in the hollow
fiber model were also found to be attenuated for survival. Shaded genes represent late-stage
survival genes detected only after 90 days of infection in the mouse lung.

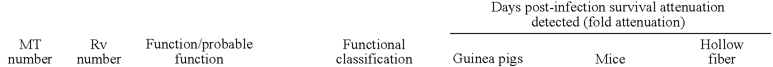

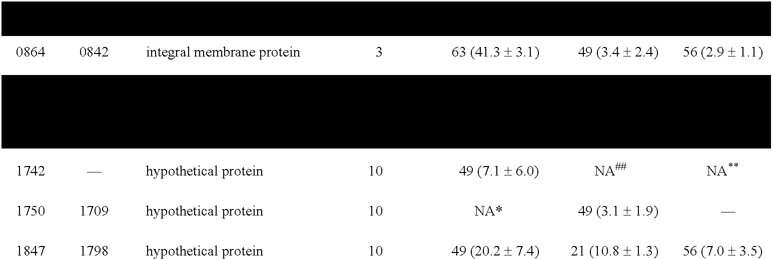

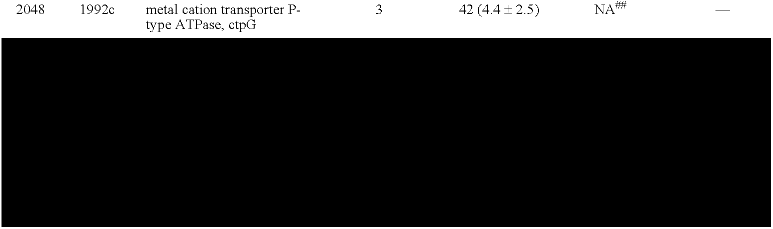

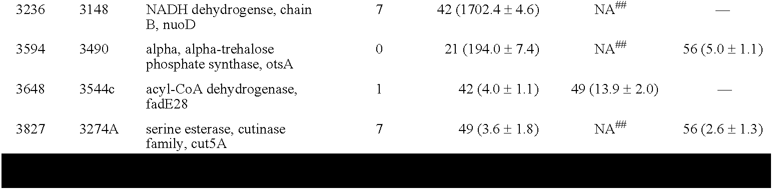

NA* = not attenuated up to day 42,
NA** = not attenuated up to day 56,
NA^{#} = not attenuated up to day 63,
NA^{##} = not attenuated up to day 360.
Fold attenuation represents mean ± standard deviation.
Functional classification key [14]: 0 virulence, detoxification, adaptation, 1 lipid metabolism, 2 information pathways, 3 cell wall and cell processes, 7 intermediary metabolism and respiration, 9 regulatory proteins, 10 conserved hypotheticals, 16 conserved hypotheticals with ortholog in M. bovis

Mutants for 9 of the 13 genes attenuated for survival in the mouse were detected only after 90 days of infection. Further, as expected, 6 of the 7 Tn mutants with Tn insertions in the distal portion of the genes were not found to be attenuated either in the guinea pig or mouse aerosol models. However, mutant JHU1742a-175 was found to be attenuated in both the guinea pig and mouse models. This mutant harbors the Tn insertion just distal (80.5%) to the 80% cutoff used in this study, and it is therefore likely that the Tn insertion was successful in inactivating this gene.

Mouse Hollow fiber infection: As proof-of-principle, selected mutants found to be attenuated for survival in either the guinea pig or mouse aerosol models were also analyzed using real-time PCR in the mouse hollow fiber model [13]. In this novel in vivo model, granulomatous lesions develop around encapsulated bacilli in semi-diffusible hollow fibers implanted subcutaneously into mice. In this microenvironment, the organisms demonstrate an altered physiologic state characterized by stationary-state colony-forming unit counts and decreased metabolic activity. Seven of the 10 mutants tested in this model, were found to be attenuated by day 56 after infection (Table 2) suggesting that these genes may be important for M. tuberculosis extracellular survival within granulomatous lesions.
Functional classification of genes represented by the M. tuberculosis Tn mutants: Table 3 summarizes the functional classification of genes represented by the M. tuberculosis Tn mutants. Mutants in 74 unique genes spanning 11 M. tuberculosis genetic functional classes were tested [14, 15]. Forty one percent were conserved hypotheticals, 16% were involved with cell wall/cell processes, 16% were involved with intermediary metabolism/respiration and the remaining 27% distributed among other functional classes. Forty two percent (5/12) of genes involved with cell wall/processes, 25% (3/12) of the genes involved with intermediary metabolism/respiration and 20% of conserved hypotheticals were found to be attenuated for survival. Thirty three percent (3/9) of the genes identified for late-stage survival (after 90 days) in the mouse lung were involved with cell wall/cell processes.

TABLE 3
Functional classification of the genes represented by the M. tuberculosis
Tn mutants.

| | M. tuberculosis Tn mutants | |
|---|---|---|
| | | Attenuated |
| Functional classification [14] | Tested | for survival |
| virulence, detoxification, adaptation | 2 | 1 |
| lipid metabolism | 6 | 1 |
| information pathways | 2 | 1 |
| cell wall and cell processes | 12 | 5 |
| insertion sequences and phages | 1 | 0 |
| PE/PPE family | 6 | 0 |
| intermediary metabolism and respiration | 12 | 3 |
| unknown | 1 | 0 |
| regulatory proteins | 2 | 1 |
| conserved hypotheticals | 24 | 5 |
| conserved hypotheticals with ortholog in | 6 | 1 |
| M. bovis | | |

Mutants in 74 unique genes spanning 11 M tuberculosis genetic functional classes were tested. Forty one percent are conserved hypotheticals, 16% are involved with cell wall/processes, 16% are involved with intermediary metabolism/respiration and the remaining 27% distributed among other functional classes. Forty two ; percent (5/12) of genes involved with cell wall/processes, 25% (3/12) of the genes involved with intermediary metabolism/respiration and 20% of conserved hypotheticals were found to be attenuated for survival.
0 virulence, detoxification, adaptation, 1 lipid metabolism, 2 information pathways, 3 cell wall and cell processes, 5 insertion sequences and phages, 6 PE/PPE, 7 intermediary metabolism and respiration, 8 unknown, 9 regulatory proteins, 10 conserved hypothetical, 16 conserved hypothetical with ortholog in M. bovis

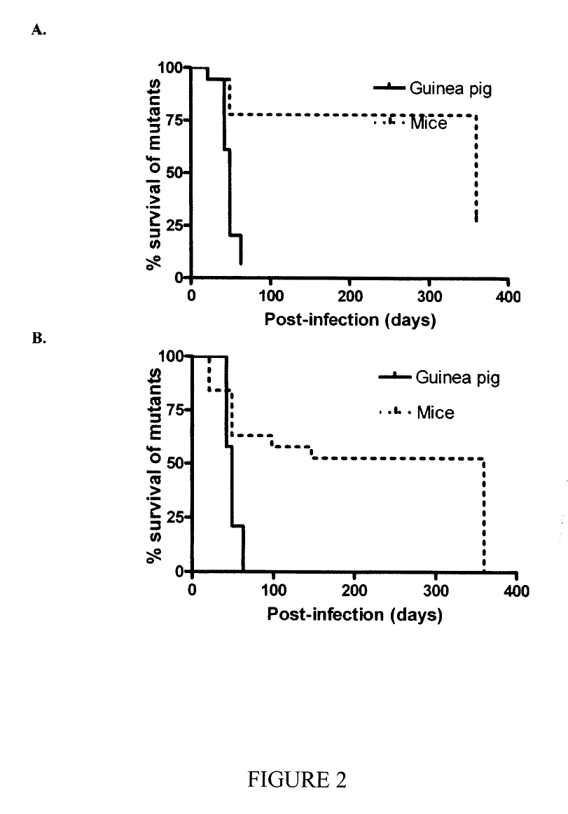

Time to detection of attenuation for survival for M. tuberculosis Tn mutants: Of the 13 M. tuberculosis Tn mutants found to be attenuated in the mouse aerosol model, less than one third (4/13) were detected by day 49. We hypothesized that the guinea pig aerosol model would be immunologically different from the mouse model due to the presence of caseous necrosis [7]. Indeed, 87% (13/15) of Tn mutants found to be attenuated in the guinea pig aerosol model were detected by day 49. FIG. 2 shows the comparison of Kaplan Meyer survival curves for the attenuated Tn mutants. Median survival times of Tn mutants in guinea pig and mouse aerosol models were 49 and 360 days, respectively (Log Rank Test p value <0.0001). When this comparison was made for the 10 Tn mutants attenuated in both the guinea pig and mouse aerosol models, median mutant survival times were still significantly shorter in the guinea pig (Log Rank Test p value=0.0003). These data show that the guinea pig model detects attenuation for survival of M. tuberculosis mutants earlier than the mouse model.

### Discussion

M. tuberculosis is an extensively host-adapted pathogen which has developed strategies to survive intracellularly (macrophages, non-professional phagocytic cells), disseminate outside of the lungs, and resist both innate and adaptive immune mechanisms, the latter of which in humans, is required for the development of caseating granulomas. At least four classes of M. tuberculosis survival defects have been described through the study of M. tuberculosis mutants in mice; these include defective growth in vivo attenuation (giv), severe growth in vivo (sgiv), persistence (per) and pathology (pat) or immunopathology (imp) phenotypes [2, 16]. However, mutants displaying these phenotypes have not been extensively characterized in animal models such as the guinea pig which form caseating necrosis as part of their adaptive immune response to M. tuberculosis. Previous studies have shown that the M. tuberculosis ΔsigC mutant was found to have a giv phenotype in guinea pigs [17] but an imp phenotype in mice [18]. Recently, Converse et al have shown that M. tuberculosis ΔdosR mutant was found to have a giv phenotype in guinea pigs, but at higher infectious doses, an imp phenotype in mice (P. Converse et al, submitted). These data suggest different bacterial survival kinetics for M. tuberculosis mutants in guinea pig and mouse models. Therefore, in this study, we compared the kinetics of bacterial survival in mammalian lungs of identical pools of M. tuberculosis mutants in two aerosol models to directly evaluate the impact of caseous necrosis seen in guinea pig tuberculosis [7] but absent in mouse tuberculosis. Further, mutants for select genes required for survival in mammalian lungs were also evaluated in the hollow-fiber dormancy mouse granuloma model.

DeADMAn is a high-throughput, subtractive identification screening tool for competitive survival of defined mutant pools. This approach has been shown to be highly sensitive for up to 100 mutants per pool [6]. In this study, we used smaller pools of up to 50 mutants, since only a limited number of bacilli can be implanted in the lungs by the aerosol route. It should be noted that DeADMAn uses pooled mutant infections which may confound results due to complementation of defective mutants by extracellular factors secreted by other mutants in the same pool. Further, since DeADMAn is an indirect measure of colony-forming-units, it is not useful for identification of imp/pat mutants. All mutants used in this study have a ΔsigF background [10]. However, since mutants were normalized against controls with the same ΔsigF background, Tn-disrupted gene rather than sigF deletion is likely responsible for the observed phenotype. Nonetheless, potential interaction of the sigF deletion with the Tn-disrupted gene cannot be completely ruled out. Finally, M. tuberculosis CDC 1551 strain was used in this study and certain animal models have shown that this strain is less virulent than the H37Rv strain [19]. However, we believe that these two strains are highly comparable. Further, since comparisons were made against controls with the same background, Tn-disrupted gene rather than the background strain is responsible for the observed phenotype. Nonetheless, it is possible that a similar analysis in the H37Rv background may result in a somewhat different list of mutants attenuated for survival than found in this study.

Eighteen M. tuberculosis genes were identified in this study to be required for survival in mammalian lungs. These include MT1847 (Rv1798) and MT3648 (Rv3544c) which have been previously described for in vivo survival in mouse lungs and spleens respectively [5,6] and MT3594 (otsA, Rv3490) whose deletion mutant has been shown to be attenuated in mouse lung and spleen after intravenous infection [20]. MT1847 (Rv1798), MT3236 (nuoD, Rv3148) and MT3594 otsA, Rv3490) have been found to be up-regulated during nutrient starvation suggesting their role in mycobacterial responses to the host environment [21]. Of the genes identified in this study, 9 were required for in vivo survival in the mouse lung beyond 90 days. We believe that these genes are required for late-stage survival and represent key virulence factors required for survival in maturing granulomatous lesions. These late-stage survival genes include MT1102 (Rv1072) and MT2050 (Rv1994c), which have been found to be up-regulated during nutrient starvation and oxidative stress [21, 22]. MT1102 (Rv1072) and MT2050 (Rv1994c) are both SigH dependent genes [16, 23], suggesting their role in adaptive responses to the host immune system. Similarly, MT0361 (Rv0346c) and MT2174 (Rv2114) were found to be up-regulated under different stress conditions including anaerobic/micro-aerophilic environment [24] and/or nutrient starvation [21] suggesting their possible role in survival against the host immune response. MT 1698 (pks10, Rv1660), is required for phthiocerol dimycocerosate (PDIM) synthesis and mutants deficient in this gene are attenuated for survival in mouse lung [25]. In addition, it is also up-regulated during nutrient starvation [21]. MT3978 (Rv3864) has been previously described to be required for in vivo survival in mouse spleens [5] and has also been found to be up-regulated during nutrient starvation [21].

Unlike mice, M. tuberculosis disease in guinea pig leads to well-formed caseous granulomas [7]. It was therefore hypothesized that mouse late-stage survival mutants may lack key virulence pathways required for survival in maturing granulomatous lesions, and the presence of caseous necrosis in guinea pig granulomas may accelerate their detection. In other words, well formed granulomas in the guinea may impose more intense, albeit similar, stressors compared to the poorly formed granulomas observed in the mouse and lead to accelerated detection of these late-stage survival mutants. Indeed, as hypothesized, the guinea pig model was found to detect attenuation for survival of majority of the Tn mutants earlier than the mouse lung model. Further, 10 mutants required for survival in mammalian lungs were evaluated in the hollow-fiber mouse granuloma model and 7 were found to be attenuated for survival in this model, suggesting that the genes represented by those mutants are important for M. tuberculosis survival within granulomatous lesions. Our hypothesis is further strengthened by the fact that several of the genes detected earliest in the guinea pig compared to mouse model [MT0361 (Rv0346c), MT1102 (Rv1072), MT2050 (Rv1994c) and MT3978 (Rv3864)] are likely to be involved with responses to stressors generated by the host immune response such as granulomas [16, 21-24].

In summary, using a competition survival assay we have identified genes required for bacterial survival in mammalian lungs after aerosol infection. There was a high degree of agreement between the genes identified by each of the models used. Long-term mouse infection showed that certain late-stage survival bacterial mutants survived for greater than 90 days, but later were out-competed by other mutants in the pools between days 150 and 360. A majority of these mouse late-stage survival mutants were identified in less than 65 days in the guinea pig model. Mouse late-stage survival mutants may lack key virulence pathways required for survival in maturing granulomatous lesions, and the presence of caseous necrosis in guinea pig granulomas may accelerate their detection.

### REFERENCES FOR BACKGROUND AND EXAMPLE 1

1. World Health Organization. 8th WHO annual report on global TB control. Global tuberculosis control—surveillance, planning, financing. Available at: http://www.who.int/tb/publications/global_report/en/. Accessed 1 Nov. 2006.
2. Hingley-Wilson S M, Sambandamurthy V K and Jacobs W R, Jr. Survival perspectives from the world's most successful pathogen, Mycobacterium tuberculosis. Nat Immunol 2003; 4:949-55.
3. Nuermberger E, Bishai W R and Grosset J H. Latent tuberculosis infection. Semin Respir Crit. Care Med 2004; 25:317-36.
4. Sassetti C M, Boyd D H and Rubin E J. Genes required for mycobacterial growth defined by high density mutagenesis. Mol Microbiol 2003; 48:77-84.
5. Sassetti C M, Rubin E J. Genetic requirements for mycobacterial survival during infection. Proc Natl Acad Sci USA 2003; 100:12989-94.
6. Lamichhane G, Tyagi S and Bishai W R. Designer arrays for defined mutant analysis to detect genes essential for survival of Mycobacterium tuberculosis in mouse lungs. Infect Immun 2005; 73:2533-40.
7. Turner O C, Basaraba R J and Orme I M. Immunopathogenesis of pulmonary granulomas in the guinea pig after infection with Mycobacterium tuberculosis. Infect Immun 2003; 71:864-71.
8. Lamichhane G, Zignol M, Blades N J, et al. A postgenomic method for predicting essential genes at subsaturation levels of mutagenesis: application to Mycobacterium tuberculosis. Proc Natl Acad Sci USA 2003; 100:7213-8.
9. Rubin E J, Akerley B J, Novik V N, Lampe D J, Husson R N and Mekalanos J J. In vivo transposition of mariner-based elements in enteric bacteria and mycobacteria. Proc Natl Acad Sci USA 1999; 96:1645-50.
10. Chen P, Ruiz R E, Li Q, Silver R F and Bishai W R. Construction and characterization of a Mycobacterium tuberculosis mutant lacking the alternate sigma factor gene, sigF. Infect Immun 2000; 68:5575-80.
11. Wiegeshaus E H, McMurray D N, Grover A A, Harding G E and Smith D W. Host-parasite relationships in experimental airborne tuberculosis. 3. Relevance of microbial enumeration to acquired resistance in guinea pigs. Am Rev Respir Dis 1970; 102:422-9.
12. Larsen M. Some Common Methods in Mycobacterial Genetics. In: Jacobs Jr W, Hatfull G, eds. Molecular Genetics of Mycobacteria. Washington, D.C.: ASM Press, 2000:316-17.
13. Karakousis P C, Yoshimatsu T, Lamichhane G, et al. Dormancy phenotype displayed by extracellular Mycobacterium tuberculosis within artificial granulomas in mice. J Exp Med 2004; 200:647-57.
14. Cole S T. TubercuList. WWW server v3.1. Available at:
   http://genolist.pasteur.fr/TubercuList/. Accessed 1 Nov. 2006.
15. TIGR. Mycobacterium tuberculosis CDC1551 Genome Page. Vol. 2006: The Institute for Genomic Research, 2005-06.
16. Kaushal D, Schroeder B G, Tyagi S, et al. Reduced immunopathology and mortality despite tissue persistence in a Mycobacterium tuberculosis mutant lacking alternative sigma factor, SigH. Proc Natl Acad Sci USA 2002; 99:8330-5.
17. Karls R K, Guarner J, McMurray D N, Birkness K A and Quinn F D. Examination of Mycobacterium tuberculosis sigma factor mutants using low-dose aerosol infection of guinea pigs suggests a role for SigC in pathogenesis. Microbiology 2006; 152:1591-600.
18. Sun R, Converse P J, Ko C, Tyagi S, Morrison N E and Bishai W R. Mycobacterium tuberculosis ECF sigma factor sigC is required for lethality in mice and for the conditional expression of a defined gene set. Mol Microbiol 2004; 52:25-38.
19. Bishai W R, Dannenberg A M, Jr., Parrish N, et al. Virulence of Mycobacterium tuberculosis CDC1551 and H37Rv in rabbits evaluated by Lurie's pulmonary tubercle count method. Infect Immun 1999; 67:4931-4.
20. Murphy H N, Stewart G R, Mischenko V V, et al. The OtsAB pathway is essential for trehalose biosynthesis in Mycobacterium tuberculosis. J Biol Chem 2005; 280:14524-9.
21. Betts J C, Lukey P T, Robb L C, McAdam R A and Duncan K. Evaluation of a nutrient starvation model of Mycobacterium tuberculosis persistence by gene and protein expression profiling. Mol Microbiol 2002; 43:717-31.
22. Schnappinger D, Ehrt S, Voskuil M I, et al. Transcriptional Adaptation of Mycobacterium tuberculosis within Macrophages: Insights into the Phagosomal Environment. J Exp Med 2003; 198:693-704.
23. Manganelli R, Voskuil M I, Schoolnik G K, Dubnau E, Gomez M and Smith I. Role of the extracytoplasmic-function sigma factor sigma(H) in Mycobacterium tuberculosis global gene expression. Mol Microbiol 2002; 45:365-74.
24. Muttucumaru D G, Roberts G, Hinds J, Stabler R A and Parish T. Gene expression profile of Mycobacterium tuberculosis in a non-replicating state. Tuberculosis (Edinb) 2004; 84:239-46.
25. Sirakova T D, Dubey V S, Cynamon M H and Kolattukudy P E. Attenuation of Mycobacterium tuberculosis by disruption of a mas-like gene or a chalcone synthase-like gene, which causes deficiency in dimycocerosyl phthiocerol synthesis. J Bacteriol 2003; 185:2999-3008.

### Introduction, Background, Significance and Preliminary Data for Examples 2-5.

The success of Mycobacterium tuberculosis as a pathogen is largely dependent on its ability to persist in the host for years or even decades. However, the microbial mechanisms involved in persistence are not well understood. We evaluated the survival of a genetically defined pool of M. tuberculosis transposon mutants in the mouse and guinea pig aerosol model. Unlike mice, M. tuberculosis-infected guinea pigs form caseating granulomas which may simulate human disease more closely. We identified mutants for 7 genes that were attenuated for survival only after 90 days of infection in the mouse but significantly earlier in the guinea pig. We hypothesize that these mouse late-stage survival mutants lack key virulence pathways required for survival in maturing granulomatous lesions and their respective disrupted genes may be targets for vaccine development against mycobacterial ‘persistence’. This proposal will further examine the roles of these genes in mycobacterial persistence and their possible use as targets for vaccine development.

Humans become infected with Mycobacterium tuberculosis via the aerosol route. Successful implantation and initial infection may lead to disease; but is frequently controlled by the host immune system, leading to ‘persistent’ infection. The microbial mechanisms involved in ‘persistence’ are not well understood and identification of M. tuberculosis genes required for ‘persistence’ may lead to the identification of key targets for drug and vaccine development [1]. Using a high-throughput subtractive identification method [2], we conducted a cross-species analysis and compared bacterial growth kinetics of identical pools of M. tuberculosis mutants in the guinea pig and mouse aerosol models. Long-term mouse infection showed that certain late-stage survival bacterial mutants survived for greater than 150 days, but later were out-competed by other mutants in the pools. The majority of these mouse late-stage survival mutants were identified in less than 65 days in the guinea pig model (FIG. 2), which unlike the mouse model displays caseous necrosis in response to tuberculosis disease [3]. Table 1 lists the genes represented by the 7 M. tuberculosis mutants identified by our studies to be attenuated significantly earlier in the guinea pig, but only after 150 days in the mouse aerosol infections [4]. Thus, these mouse late-stage survival mutants probably lack key virulence pathways required for survival in maturing granulomatous lesions should serve as excellent targets for vaccine development against mycobacterial ‘persistence’. This proposal will further examine the roles of these mouse late-stage survival genes in mycobacterial persistence and their possible use as vaccines using recent advances in vaccine technologies [5] and as drug targets for rational design of anti-tuberculosis drugs.

### Example 2

Testing of the attenuation for survival of each of the 7 mouse late-stage survival mutants and their complemented strains as individual infections in the guinea pig and mouse aerosol models.

### Research Design and Methods

Genes represented by 7 mouse late-stage survival mutants (Table 4) are part of key virulence pathways required for bacterial persistence and survival in maturing granulomatous lesions.

TABLE 4
Seven M. tuberculosis mutants attenuated significantly earlier in guinea pig,
but only after 150 days in mouse aerosol infections. Fold attenuation was measured
by real time PCR analysis. Predicted T cell epitopes per 100 aa was compared to
ESAT-6 and CFP-10 which had 95 and 84 epitopes/100 aa, respectively.

| | | | | Attenuated for survival | |
|---|---|---|---|---|---|
| | | | Predicted T | detected days post- | |
| | | | cell | infection (fold attenuation) | |
| MT | Rv | Function/probable | epitopes/ | Guinea | |
| number | number | function | 100 aa | Pig | Mouse |
| 0361 | 0346c | L-asparagine permease, | 169 | 42 | 360 |
| | | ansP2 | | (9.2 ± 5.7) | (3.5 ± 1.5) |
| 1102 | 1072 | transmembrane protein | 151 | 49 | 360 |
| | | | | (9.4 ± 5.8) | (14.6 ± 1.1) |
| 2050 | 1994c | transcriptional | 83 | 42 | 360 |
| | | regulatory protein | | (58.4 ± 1.8) | (39.4 ± 2.0) |
| 2500 | 2427c | gamma-glutamyl | 89 | 63 | 360 |
| | | phosphate reductase, | | (4.3 ± 1.4) | (8.6 ± 2.5) |
| | | proA | | | |
| 2884 | 2817c | hypothetical protein | 107 | 49 | 360 |
| | | | | (2.9 ± 1.7) | (3.1 ± 2.1) |
| 3097 | 3017c | ESAT-6 like protein, | 88 | 42 | 360 |
| | | esxQ | | (38.5 ± 1.1) | (58.4 ± 4.8) |
| 3978 | 3864 | hypothetical protein | 113 | 49 | 360 |
| | | | | (3.2 ± 1.7) | (7.8 ± 1.3) |

Background: Mouse late-stage survival mutants were identified using a high-throughput competitive survival assay (DeADMAn). However, this approach may confound results due to complementation of defective mutants by extracellular factors secreted by other mutants in the same pool. To verify the phenotype of these mutants and to confirm that the disrupted gene was indeed the reason for the observed phenotype, we propose to test the attenuation for survival for each of the 7 mouse late-stage survival mutants and their complemented strains as individual infections in the guinea pig and mouse aerosol models.
Construction of the complemented strain: Each of the 7 genes with their native promoter (600 bp proximal to the gene) are cloned into the modified single copy integrating E. coli-mycobacteria shuttle plasmid pMH94 (integrates at the mycobacterial attB site) with a zeocin marker. This construct is used to transform each of the respective M. tuberculosis Tn mutants using standard mycobacterial methods [6]. Integration is confirmed by Southern blotting and PCR.
Aerosol infection: M. tuberculosis Tn mutants and their respective complemented strains are grown to mid-log phase and are used for guinea pig and mouse aerosol infections as described previously [4]. Briefly, each mutant and its complemented strain is grown separately at 37° C. in Middlebrook 7H9 media (Becton Dickinson, Sparks, Md.) supplemented with O-ADC (Becton Dickinson, Sparks, Md.), 5% glycerol, and kanamycin at 20 μg/ml. The survival of the parent M. tuberculosis strain is compared to each of the mutant and its complemented strain.
Guinea pig infection: 250-300 gm Hartley strain guinea pigs (Charles River, Wilmington, Mass.) are infected via the aerosol route using the latest version of the Madison Aerosol Chamber from the University of Wisconsin Engineering Shops [7]. Four guinea pigs are used for each time point for each of the 15 groups (7 mutants, 7 complemented; 1 parent strain control) and are sacrificed at days 1, 21 and 49 and 63 post-infection. Both the lungs in entirety are homogenized in phosphate-buffered saline (PBS) and the entire homogenate is plated on Middlebrook 7H11 solid medium (Becton Dickinson, Sparks, Md.) with kanamycin at 20 μg/ml to determine CFU counts. Survival of each mutant in the lungs is compared to the parent strain and the complemented mutant.
Mouse infection: 5-6 week-old female BALB/c mice (Charles River, Wilmington, Mass.) are infected via the aerosol route using the Glas-Col inhalation exposure system (Glas-Col, Terre Haute, Ind.). Four mice pigs are used for each time point for each of the 15 groups (7 mutants, 7 complemented; 1 parent strain control) and are sacrificed at days 1, 21, 49, 96 147 and 360 post-infection. The survival of each mutant in the lungs is analyzed. Both the lungs in entirety are homogenized in phosphate-buffered saline (PBS) and the entire homogenate is plated on Middlebrook 7H11 solid medium (Becton Dickinson, Sparks, Md.) with kanamycin at 20 μg/ml to determine CFU counts. Survival of each mutant in the lungs is compared to the parent strain and the complemented mutant.

All plates are incubated at 37° C. for at least 3 weeks before the colonies are counted. Standard statistical tests are used to evaluate the comparisons among the various groups. The results show that M. tuberculosis mutants are attenuated for survival in the mouse aerosol model after 150 days of infection but by day 63 in the guinea pig model. Further, the results show that the respective complementation strains have a phenotype similar to the parent M. tuberculosis strain, suggesting that the disrupted gene is indeed the reason for the observed phenotype.

If Tn insertion confers a polar effect on the downstream gene(s) and therefore complementation is not successful in reverting to the parent phenotype, then complemented strains are generated with these downstream genes and evaluated in these models. Survival and growth characteristics of the transposon mutants and their complemented strains are expected to be similar to their parent strain, and are compared prior to these experiments.

### Example 3

### Testing of T Cell Responses against Recombinant Proteins for the 7 Genes by Measuring Interferon-γ Production from Antigen Stimulated Lymphocytes Derived from Individuals with Latent Tuberculosis

This experiment will show that T cell responses develop against recombinant proteins for these 7 genes during latent tuberculosis.

Background: Four of the 7 genes identified code for membrane proteins or are secreted extracellularly. Further, there are many examples of antibodies in convalescent sera which are directed to cytoplasmic proteins. [8, 9] Therefore, some of the proteins encoded by these 7 genes are immunogenic and elicit T cell responses in the human host during latent tuberculosis. Bio-informatics analysis (Table 4) shows that these 7 proteins have comparable number of human MHC II T cell epitopes per 100 aa when compared with ESAT-6 and CFP-10 [10]. Therefore interferon-γ production from human lymphocytes (derived from individuals with latent tuberculosis) in response to challenge by recombinant proteins for these 7 M. tuberculosis genes is measured.
Research design: Generation of recombinant protein: Coding sequences for the 7 genes are amplified from M. tuberculosis CDC 1551 genomic DNA by polymerase chain reaction and are cloned into the recombinant protein expression vector pET-22b(+) (Novagen, San Diego, Calif.) as a C-terminal hexa-histidine tag fusion. After confirmation of in-frame fusion by sequencing, the recombinant proteins are expressed in E. coli BL21(DE3) and are purified using metal affinity chromatography using standardized techniques.
Lymphocytes from individuals with latent tuberculosis: Peripheral blood specimens are obtained from 5-10 PPD positive healthy volunteers. Lymphocytes are separated using standard protocols and incubated with recombinant proteins for each of the genes above in triplicate. Human interferon-γ ELISPOT (eBioscience, San Diego, Calif.) is used to measure the interferon-γ production. Un-stimulated lymphocytes are used as controls.

Proteins containing hydrophobic or membrane-associated domains and which partition into the insoluble fraction of standard lysis buffer are solubilized using commercially available formulations utilizing nonionic and zwitterionic detergents (BugBuster® Protein Extraction Reagent, PopCulture® Reagent), which are capable of solubilizing membrane bound or hydrophobic proteins without denaturation.

Mycobacterial proteins that are not expressed in E. coli are instead expressed in M. smegmatis as described by Choudhuri et al [11].

### Example 4

### Evaluation of Vaccines

### Research Design:

a. DNA Vaccines:

A number of M. tuberculosis genes have been identified that, when mutated, result in attenuation of mycobacterial persistence in both mouse and guinea pig models of infection (Table 5). These genes therefore represent important virulence factors required for persistence of this pathogen within a host and may be used as targets for vaccine development. Since DNA vaccines can induce substantial cellular immunity and evoke both CD4+ and CD8+ T-cell responses, this approach engendesr protection from M. tuberculosis infection that is equivalent to, or better than, the current BCG regimen. DNA vaccines that separately express each of the 7 persistence-associated genes from M. tuberculosis (Table 4) are created, and tested in mice and guinea pigs for both their immunogenicity, as well as their ability to confer protection from an aerosol challenge with a virulent strain of M. tuberculosis.

TABLE 5
Recently identified genes associated
with persistence of M. tuberculosis

| Rv number | Function/probable function |
|---|---|
| 0346c | L-asparagines permease, ansP2 |
| 1072 | Transmembrane protein |
| 1994c | Transcriptional regulatory protein |
| 2427c | Gamma-glutamyl phosphate reductase, proA |
| 2817c | Hypothetical protein |
| 3017c | ESAT-6-like protein, esxQ |
| 3864 | Hypothetical protein |

To test DNA vaccines that incorporate the genes listed above, each gene is cloned into a pJW4303 (or similar) plasmid vector, grown to sufficient quantity and endotoxin-free plasmid DNA is purified.

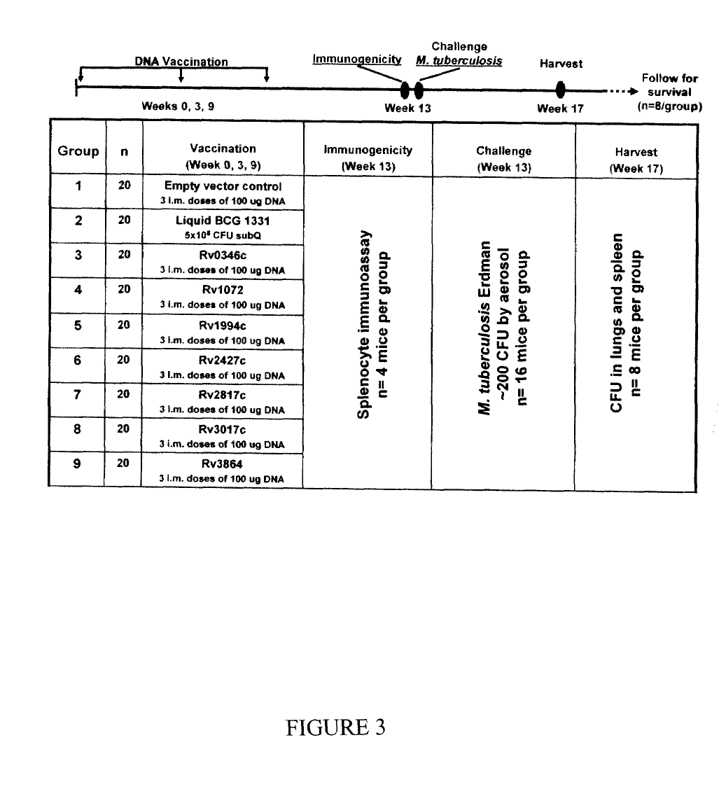

I. For in vivo evaluation of these vaccines, female 6-8 week-old BALB/c mice (Charles River, Wilmington, Mass.) (20 animals per group) are vaccinated intramuscularly with 3 doses of 100 μg DNA delivered at 3 week intervals (FIG. 3). One group of mice (Group 1, FIG. 3) is immunized with plasmid DNA that lacks a mycobacterial gene using the same dose and schedule and represents the “negative control” group. Another group (Group 2, FIG. 2) is vaccinated subcutaneously once at week 0 with a standard reference strain of BCG and serves as the “positive control” group. The protective efficacy of the candidate DNA vaccines is determined by comparison to the protection elicited by this BCG standard.

Four weeks after the last immunization (i.e. study week 13) the immunogenicity of these vaccine regimens is determined by immunoassays that measure IFN-γ production since this cytokine is critical for protection from tuberculosis. Four mice from each group are sacrificed and their spleens are removed. Cell suspensions are made from each spleen and the resulting splenocytes are tested for antigen-specific immune responses. One set of cells is cultured for 3 days with M. tuberculosis proteins (including purified protein derivative, culture filtrate protein and recombinant proteins encoded by the genes incorporated into the vaccine vectors) and the amount of IFN-γ in the culture supernatant is measured by ELISA after 3 days. A 2^{nd} set of splenocytes is stimulated for 6 hours with peptides derived from M. tuberculosis antigens and is then stained for intracellular IFN-γ and analyzed by flow cytometry.

The remaining mice (16 animals per group) are then aerogenically challenged with ˜200 CFU of the virulent CDC1551 (or Erdman) strain of M. tuberculosis using the Glas-Col inhalation exposure system (Glas-Col, Terre Haute, Ind.). After 4 weeks (i.e. study week 17) 8 mice per group are sacrificed and their lungs and spleens are removed. A portion of each organ is stained with hematoxylin and eosin to evaluate histopathology. The remaining tissue is homogenized and plated on agar to determine the bacterial burden in each of these organs. The remaining 8 mice per group are followed for survival to establish mean survival times. Both immunogenicity and protection data are analyzed using standard statistical tests to evaluate these novel DNA vaccines that incorporate these genes associated with mycobacterial persistence.

II. 250-300 gm Hartley strain guinea pigs (Charles River, Wilmington, Mass.) (16 animals per group) are vaccinated intramuscularly with 3 doses of 100 μg DNA delivered at 3 week intervals. One group of guinea pigs is immunized with plasmid DNA that lacks a mycobacterial gene using the same dose and schedule and represents the “negative control” group. Another group is vaccinated subcutaneously once at week 0 with a standard reference strain of BCG and serve as the “positive control” group. The protective efficacy of the candidate DNA vaccines is determined by comparison to the protection elicited by this BCG standard.

Four weeks after the last immunization (i.e. study week 13) the guinea pigs are aerogenically challenged with ˜5-10 CFU of the virulent CDC1551 (or Erdman) strain of M. tuberculosis using the latest version of the Madison Aerosol Chamber from the University of Wisconsin Engineering Shops [7]. After 4 weeks (i.e. study week 17), 8 guinea pigs per group are sacrificed and their lungs and spleens are removed. A portion of each organ is stained with hematoxylin and eosin to evaluate histopathology. The remaining tissue is homogenized and plated on agar to determine the bacterial burden in each of these organs. The remaining 8 guinea pigs per group are followed for survival to establish mean survival times. Both immunogenicity and protection data are analyzed using standard statistical tests to evaluate these novel DNA vaccines that incorporate these genes associated with mycobacterial persistence.

b. Recombinant BCG with Over-Expressing the Listed Target Gene:

Coding regions for each of these 7 genes are PCR amplified and cloned individually into the mycobacterial-E. coli shuttle vector pMV261 carrying hsp60—a strong constitutively expressed mycobacterial promoter [12]. BCG 1331 is then transformed with each of these plasmids individually to obtain 7 different recombinant BCG strains.

Seven groups of 250-300 gm Hartley strain guinea pigs (Charles River, Wilmington, Mass.) (16 animals per group) are vaccinated with the recombinant BCG strain (1 strain per group) via subcutaneous or aerosol route [13]. One group of guinea pigs is vaccinated subcutaneously once at week 0 with a standard reference strain of BCG and serves as the “positive control” group. The protective efficacy of the candidate DNA vaccines is determined by comparison to the protection elicited by this BCG standard.

Six weeks after immunization, the guinea pigs are aerogenically challenged with ˜5-10 CFU of the virulent CDC1551 strain (or Erdman) of M. tuberculosis using the latest version of the Madison Aerosol Chamber from the University of Wisconsin Engineering Shops [7]. After 4 weeks (i.e. study week 10), 8 guinea pigs per group are sacrificed and their lungs and spleens are removed. A portion of each organ is stained with hematoxylin and eosin to evaluate histopathology. The remaining tissue is homogenized and plated on agar to determine the bacterial burden in each of these organs. The remaining 8 guinea pigs per group are followed for survival to establish mean survival times. Both immunogenicity and protection data are analyzed using standard statistical tests to evaluate these novel DNA vaccines that incorporate these genes associated with mycobacterial persistence.

c. Evaluation of M. tuberculosis Persistence Gene Mutants as Vaccine Candidates in the Guinea Pig Aerosol Challenge Model.

A number of M. tuberculosis genes have been identified that, when mutated, result in attenuation of mycobacterial persistence in both mouse and guinea pig models of infection (Table 6). These genes represent important virulence factors required for persistence of this pathogen within the host and mycobacteria incapable of producing these critical genes serve as attenuated live vaccines.

TABLE 6
Recently identified genes associated
with persistence of M. tuberculosis

| Rv number | Function/probable function |
|---|---|
| 0346c | L-asparagines permease, ansP2 |
| 1072 | Transmembrane protein |
| 1994c | Transcriptional regulatory protein |
| 2427c | Gamma-glutamyl phosphate reductase, proA |
| 2817c | Hypothetical protein |
| 3017c | ESAT-6-like protein, esxQ |
| 3864 | Hypothetical protein |

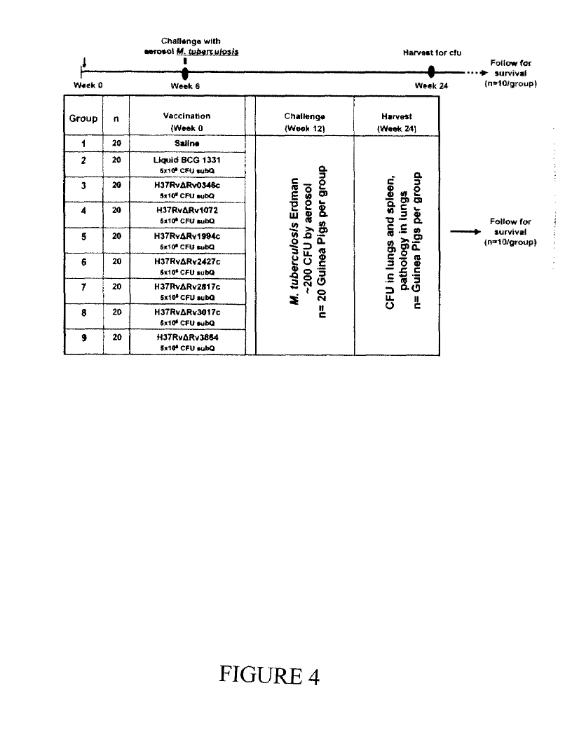

To demonstrate this conclusion, the M. tuberculosis mutants described, i.e. the seven M. tuberculosis mutant strains incapable of producing either Rv0346c, Rv1072c, Rv1994c, Rv2427c, Rv2817c, Rv3017c, or Rv3864 are used to vaccinate 7 groups of guinea pigs (FIG. 4). A negative control group of guinea pigs (Group 1, FIG. 3) are immunized with saline and a positive control group (Group 2, FIG. 3) is vaccinated subcutaneously with BCG Danish 1331.

Six weeks after immunization, the guinea pigs are aerogenically challenged with ˜5-10 CFU of the virulent CDC1551 (or Erdman) strain of M. tuberculosis using the latest version of the Madison Aerosol Chamber from the University of Wisconsin Engineering Shops [7]. After 4 weeks (i.e. study week 10), 10 guinea pigs per group are sacrificed and their lungs and spleens are removed. A portion of each organ is stained with hematoxylin and eosin to evaluate histopathology. The remaining tissue is homogenized and plated on agar to determine the bacterial burden in each of these organs. The remaining 10 animals per group are followed for survival to establish mean survival times and killing curves. Both histopathology and protection data are analyzed using standard statistical tests to evaluate the protective efficacy of these attenuated M. tuberculosis vaccine strains.

If vaccine strains (recombinant BCG strains and mutants for the late-stage genes) grown on plates with wild-type M. tuberculosis cultures at the designated primary end-points overestimate the CFU counts, the lung and spleen homogenates are also plated on kanamycin selection plates where only vaccine strains will grow. The final CFU counts are then determined by subtracting CFU counts obtained from kanamycin plates from the CFU counts for the wild-type M. tuberculosis.

### Example 5

### Evaluation of the Potential of the 7 Mouse Late-Stage Genes as Drug Targets

Background: The need for new drugs against tuberculosis is urgent in order to effectively treat disease and reduce transmission of M. tuberculosis. The rapid emergence of multiple- and extensively-drug resistant strains of M. tuberculosis has made the disease one of highest public health priorities, especially in the developing world. Existing drugs have become ineffective in many settings due to the requirement for protracted periods of treatment and the emergence of resistance. Therefore, new and effective drugs to treat infected individuals and to prevent transmission of M. tuberculosis are essential for global health. Future drugs for TB are likely to be identified by rational drug design in which essential genes are targeted and advanced structural modeling is used to generate inhibitors. The 7 genes identified in Table 7 serve as drug targets for rational design of anti-tuberculosis drugs.

TABLE 7
Recently identified genes associated
with persistence of M. tuberculosis

| Rv number | Function/probable function |
|---|---|
| 0346c | L-asparagines permease, ansP2 |
| 1072 | Transmembrane protein |
| 1994c | Transcriptional regulatory protein |
| 2427c | Gamma-glutamyl phosphate reductase, proA |
| 2817c | Hypothetical protein |
| 3017c | ESAT-6-like protein, esxQ |
| 3864 | Hypothetical protein |

Research design: Generation of recombinant protein: Coding sequences for these 7 genes are amplified from M. tuberculosis CDC 1551 genomic DNA by polymerase chain reaction and are cloned into the recombinant protein expression vector pET-22b(+) (Novagen, San Diego, Calif.) as a C-terminal hexa-histidine tag fusion. After confirmation of in-frame fusion by sequencing, the recombinant proteins are expressed in E. coli BL21(DE3) and purified using metal affinity chromatography using standardized techniques. These recombinant proteins are bio-chemically evaluated for their function e.g. ATPase, protease, esterase, hydrolase, dehydrogenase, etc. A library of molecules is tested and molecules that inhibit the bio-chemical function of the recombinant proteins are identified. Further analysis as to the affinity of binding, solubility, likely penetration of tissue, etc is also done.

If proteins containing hydrophobic or membrane-associated domains partition into the insoluble fraction of standard lysis buffer, then commercially available formulations utilizing nonionic and zwitterionic detergents (BugBuster® Protein Extraction Reagent, PopCulture® Reagent) are used to solubilize those proteins without denaturation.

Mycobacterial proteins that are not expressed in E. coli are expressed in M. smegmatis as described by Choudhuri et al [11].

### REFERENCES FOR EXAMPLES 2-5

1. Hingley-Wilson S M, Sambandamurthy V K and Jacobs W R, Jr. Survival perspectives from the world's most successful pathogen, Mycobacterium tuberculosis. Nat Immunol 2003; 4:949-55
2. Lamichhane G, Tyagi S and Bishai W R. Designer arrays for defined mutant analysis to detect genes essential for survival of Mycobacterium tuberculosis in mouse lungs. Infect Immun 2005; 73:2533-40
3. Turner O C, Basaraba R J and Orme I M. Immunopathogenesis of pulmonary granulomas in the guinea pig after infection with Mycobacterium tuberculosis. Infect Immun 2003; 71:864-71
4. Jain S K, Hernandez-Abanto S, Cheng Q, et al. Accelerated detection of Mycobacterium tuberculosis genes essential for bacterial survival in guinea pigs compared with mice. J Infect Dis. (in press)
5. Skeiky Y A, Sadoff J C. Advances in tuberculosis vaccine strategies. Nat Rev Microbiol 2006; 4:469-76
6. Larsen M. Some Common Methods in Mycobacterial Genetics. In: Jacobs Jr W, Hatfull G, eds. Molecular Genetics of Mycobacteria. Washington, D.C.: ASM Press, 2000:317
7. Wiegeshaus E H, McMurray D N, Grover A A, Harding G E and Smith D W. Host-parasite relationships in experimental airborne tuberculosis. 3. Relevance of microbial enumeration to acquired resistance in guinea pigs. Am Rev Respir Dis 1970; 102:422-9
8. Havlasova J, Hernychova L, Halada P, et al. Mapping of immunoreactive antigens of Francisella tularensis live vaccine strain. Proteomics 2002; 2:857-67
9. Vytvytska O, Nagy E, Bluggel M, et al. Identification of vaccine candidate antigens of Staphylococcus aureus by serological proteome analysis. Proteomics 2002; 2:580-90
10. Peters B, Sidney J, Bourne P, et al. The immune epitope database and analysis resource: from vision to blueprint. PLoS Biol 2005; 3:e91
11. Choudhuri B S, Bhakta S, Barik R, Basu J, Kundu M and Chakrabarti P. Overexpression and functional characterization of an ABC (ATP-binding cassette) transporter encoded by the genes drrA and drrB of Mycobacterium tuberculosis. Biochem J 2002; 367:279-85
12. Stover C K, de la Cruz V F, Fuerst T R, et al. New use of BCG for recombinant vaccines. Nature 1991; 351:456-60
13. Nuermberger E L, Yoshimatsu T, Tyagi S, Bishai W R and Grosset J H. Paucibacillary tuberculosis in mice after prior aerosol immunization with Mycobacterium bovis BCG. Infect Immun 2004; 72:1065-71

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

1. A composition suitable for vaccination or eliciting an immune response, comprising
a. at least one recombinant protein or antigenic fragment thereof, wherein said at least one recombinant protein is encoded by:
a gene represented by
i) an Mtb CDC 1551 genome MT number selected from the group consisting of 0361, 1102, 2050, 2500 and 2884, or
ii) an Mtb H37Rv genome Rv number selected from the group consisting of 0346c, 1072, 1994c, 2427c, and 2817c; or
a homologous gene corresponding to i) or ii);
or
b. at least one nucleic acid molecule encoding said at least one recombinant protein or antigenic fragment thereof;
or
c. a host harboring at least one of said nucleic acid molecules.

2. The composition of claim 1, wherein said at least one nucleic acid is part of a vector.

3. The composition of claim 2, wherein said vector is selected from the group consisting of plasmids and viral vectors.

4. The composition of claim 1, wherein said host includes said at least one nucleic acid molecule incorporated into its genome or as a plasmid.

6. The composition of claim 1 further comprising a physiologically compatible carrier.

7. The composition of claim 1, wherein said host is a bacterium.

8. A composition that vaccinates against persistent mycobacterial infection, comprising
a. at least one recombinant protein or antigenic fragment thereof, wherein said at least one recombinant protein is encoded by:
a gene represented by
i) an Mtb CDC1551 genome MT number selected from the group consisting of 0361, 1102, 2050, 2500, 2884, 3097 and 3978, or
ii) an Mtb H37Rv genome Rv number selected from the group consisting of 0346c, 1072, 1994c, 2427c, 2817c, 3017c and 3864; or
a homologous gene corresponding to i) or ii);
or
b. at least one nucleic acid molecule encoding said at least one recombinant protein or antigenic fragment thereof;
or
c. a host harboring at least one of said nucleic acid molecules.

9. The composition of claim 8, wherein said at least one nucleic acid is part of a vector.

10. The composition of claim 9, wherein said vector is selected from the group consisting of plasmids and viral vectors.

11. The composition of claim 8, wherein said host includes said at least one nucleic acid molecule incorporated into its genome or as a plasmid.

12. The composition of claim 8 further comprising a physiologically compatible carrier.

13. The composition of claim 8, wherein said host is a bacterium.

14. A method of vaccinating an individual against persistent mycobacterial infection, comprising the step of
administering to said individual a vaccine composition, comprising
a. at least one recombinant protein or antigenic fragment thereof, wherein said at least one recombinant protein is encoded by:
a gene represented by
i) an Mtb CDC1551 genome MT number selected from the group consisting of 0361, 1102, 2050, 2500, 2884, 3097 and 3978, or
ii) an Mtb H37Rv genome Rv number selected from the group consisting of 0346c, 1072, 1994c, 2427c, 2817c, 3017c and 3864; or
a homologous gene corresponding to i) or ii);
or
b. at least one nucleic acid molecule encoding said at least one recombinant protein or antigenic fragment thereof,
or
c. a host harboring at least one of said nucleic acid molecules.

15. The method of claim 14, wherein said at least one nucleic acid is part of a vector.

16. The method of claim 15, wherein said vector is selected from the group consisting of plasmids and viral vectors.

17. The method of claim 14, wherein said host includes said at least one nucleic acid molecule incorporated into its genome or as a plasmid.

18. The method of claim 14 further comprising a physiologically compatible carrier.

19. The method of claim 14, wherein said host is a bacterium.

20. The method of claim 14, wherein said step of administering is carried out intravenously or by administering an aerosol.

21. An attenuated, non-pathogenic mycobacteria, wherein said attenuated mycobacteria contains at least one mutation in at least one gene selected from the group consisting of
i) an Mtb CDC1551 genome MT number selected from the group consisting of 0361, 1102, 2050, 2500, 2884, 3097 and 3978;
ii) an Mtb H37Rv genome Rv number selected from the group consisting of 0346c, 1072, 1994c, 2427c, 2817c, 3017c and 3864; and
iii) a homologous gene corresponding to i) or ii);
wherein said at least one mutation causes a reduction or elimination of a biological activity or function of a protein or polypeptide encoded by said at least one gene.

22. The method of claim 21, wherein said mycobacteria is Mycobacterium tuberculosis.

23. A method of vaccinating an individual against Mycobacterium infection, comprising the step of
administering to said individual a composition comprising
an attenuated, non-pathogenic mycobacteria, wherein said attenuated mycobacteria contains at least one mutation in at least one gene selected from the group consisting of
i) an Mtb CDC1551 genome MT number selected from the group consisting of 0361, 1102, 2050, 2500, 2884, 3097 and 3978;
ii) an Mtb H37Rv genome Rv number selected from the group consisting of 0346c, 1072, 1994c, 2427c, 2817c, 3017c and 3864; and
iii) a homologous gene corresponding to i) or ii);
wherein said at least one mutation causes a reduction or elimination of a biological activity or function of a protein or polypeptide encoded by said at least one gene.

24. The method of claim 23, wherein said Mycobacterium is Mycobacterium tuberculosis.

25. A pharmacological agent that inhibits or prevents a biological activity or function of a protein or polypeptide encoded by a gene represented by
i) an Mtb CDC1551 genome MT number selected from the group consisting of 0361, 1102, 2050, 2500, 2884, 3097 and 3978; or
ii) an Mtb H37Rv genome Rv number selected from the group consisting of 0346c, 1072, 1994c, 2427c, 2817c, 3017c and 3864; or
iii) a homologous gene corresponding to i) or ii).

26. A method of treating a Mycobacterium infection in a patient in need thereof, comprising the step of
administering to said patient a pharmacological agent that inhibits or prevents a biological activity or function of a protein or polypeptide encoded by a gene represented by
i) an Mtb CDC1551 genome MT number selected from the group consisting of 0361, 1102, 2050, 2500, 2884, 3097 and 3978; or
ii) an Mtb H37Rv genome Rv number selected from the group consisting of 0346c, 1072, 1994c, 2427c, 2817c, 3017c and 3864; or
iii) a homologous gene corresponding to i) or ii).

27. A method of vaccinating an individual against Mycobacterium infection, comprising the step of
administering to said individual a vaccine composition, comprising
a. at least one recombinant protein or antigenic fragment thereof, wherein said at least one recombinant protein is encoded by:
a gene represented by
i) an Mtb CDC1551 genome MT number selected from the group consisting of 0361, 1102, 2050, 2500 and 2884, or
ii) an Mtb H37Rv genome Rv number selected from the group consisting of 0346c, 1072, 1994c, 2427c, and 2817c; or
a homologous gene corresponding to i) or ii);
or
b. at least one nucleic acid molecule encoding said at least one recombinant protein or antigenic fragment thereof;
or
c. a host harboring at least one of said nucleic acid molecules.

28. The method of claim 27, wherein said at least one nucleic acid is part of a vector.

29. The method of claim 28, wherein said vector is selected from the group consisting of plasmids and viral vectors.

30. The method of claim 27, wherein said host includes said at least one nucleic acid molecule incorporated into its genome or as a plasmid.

31. The method of claim 27 further comprising a physiologically compatible carrier.

32. The method of claim 27, wherein said host is a bacterium.

33. The method of claim 27, wherein said step of administering is carried out intravenously or by administering an aerosol.

34. A method for identifying drugs for prevention or treatment of infections caused by Mycobacterium species, comprising the steps of
exposing a candidate drug to a recombinant protein encoded by:
a gene represented by
i) an Mtb CDC1551 genome MT number selected from the group consisting of 0361, 1102, 2050, 2500 and 2884, or
ii) an Mtb H37Rv genome Rv number selected from the group consisting of 0346c, 1072, 1994c, 2427c, and 2817c; or
a homologous gene corresponding to i) or ii);
and
determining whether or not said candidate drug inhibits a biological activity or function of said recombinant protein; and
selecting candidate drugs that inhibit said biological activity or function as drugs for prevention or treatment of infections caused by Mycobacterium species.


SEQUENCE LISTING (14 sequences)

SEQ ID NO: 1 - DNA, 1464 bp, Mycobacterium tuberculosis
atgccaccgctagacatcaccgacgagcggctcacgcgcgaagacacgggctatcacaaaggcctccatagccggcaactgcaaatgatcgctctcggtggtgccatcggtaccggcctttttctcggcgccggcggccgccttgcatcggcggggcccggtttgttcctggtctatgggatctgcggcatcttcgtgttcctgatcctgcgcgcgctcggcgaactggtgctgcaccgtccgtcctcggggtcattcgtgtcctacgcccgcgagttctacggggaaaaggtggccttcgtcgccgggtggatgtacttcctgaactgggcgatgacggggatcgtcgataccaccgcgatcgcgcactattgccattactggcgggcgtttcaacccatcccccagtggacgctggcgttgatcgcgctgctggtcgtgttgtcgatgaacctgatctcggtcaggctcttcggcgagctggagttctgggcctcattgatcaaggtgatcgcgctggttacgtttctgatcgtcggtacggtctttctcgctgggcgctacaagatcgacggccaggagaccggggtctccctgtggagcagccatggaggcatcgtgccaaccggtctgctgcccatagtgctggtcacctcgggggtggtattcgcctacgccgcaatcgaactggtgggcatcgcggccggggaaacagccgaaccggcaaagatcatgccgcgcgcgatcaactctgtggtgttgcgcatcgcgtgcttctacgtcgggtcgactgtcctgctggccctgctgctgccctataccgcctacaaagaacatgtcagcccgttcgtgaccttcttttccaagatcggtatcgacgcggcgggcagcgtgatgaacctcgtcgtgctcaccgccgcgctgtcgagcctcaacgccggactgtattccaccggacggatcctgcgatcaatggcgatcaacggcagcggcccgaggttcaccgcaccaatgtcgaagaccggcgtgccgtacggcgggatcctgctcaccgccggtatcggcctgttgggcatcattctcaacgccatcaaaccgagccaagccttcgagatcgtgctccacatcgccgcgacgggagtcatcgcggcctgggcgacgatcgtggcttgccagctgcggctccaccggatggcgaacgccgggcagctgcagcggccgaaattccggatgccgctttcaccgttcagcggctatctgacgctggctttcctggccggcgtgctgatcctgatgtacttcgacgaacaacacggtccctggatgatcgccgcaacggtgatcggtgtaccggccctcatcggtggctggtatttggtccgcaatcgtgtgacggccgtcgcgcaccacgccatcgaccacaccaagagtgtcgcggtcgtccattcggcggatccaatctga

SEQ ID NO: 2 - protein, 487 aa, Mycobacterium tuberculosis
MPPLDITDERLTREDTGYHKGLHSRQLQMIALGGAIGTGLFLGAGGRLASAGPGLFLVYGICGIFVFLILRALGELVLHRPSSGSFVSYAREFYGEKVAFVAGWMYFLNWAMTGIVDTTAIAHYCHYWRAFQPIPQWTLALIALLVVLSMNLISVRLFGELEFWASLIKVIALVTFLIVGTVFLAGRYKIDGQETGVSLWSSHGGIVPTGLLPIVLVTSGVVFAYAAIELVGIAAGETAEPAKIMPRAINSVVLRIACFYVGSTVLLALLLPYTAYKEHVSPFVTFFSKIGIDAAGSVMNLVVLTAALSSLNAGLYSTGRILRSMAINGSGPRFTAPMSKTGVPYGGILLTAGIGLLGIILNAIKPSQAFEIVLHIAATGVIAAWATIVACQLRLHRMANAGQLQRPKFRMPLSPFSGYLTLAFLAGVLILMYFDEQHGPWMIAATVIGVPALIGGWYLVRNRVTAVAHHAIDHTKSVAVVHSADPI

SEQ ID NO: 3 - DNA, 837 bp, Mycobacterium tuberculosis
gtgcgggagacaagcaacccggtatttcgttcgttgcctaagcagcggggcggatacgcgcaattcggaactggcaccgcccagcagggattcccagccgatccctacctggcgccctatcgggaagcaaaggccacccgcccgctgaccatcgacgatgtcgtgaccaagacgggcctgacgctggctatgttggcgggcaccgccgtcgtctcctacttcctggttgcgtcgaacgtcgcactggccatgccgctgaccttggtgggggctttgggtggtttggcgctggtgctggtggccaccttcggccgcaagcaggacaacccggcgatcgtgctcagctacgcggcgctcgagggcctgttcctgggtgccatctcgttcgtcttggctaacttcacggtggcgtccgcgaatgctggggtgctgatcggggaggccatcttagggacgatgggtgtgttcttcggcatgctcgtcgtctacaagacaggggccatccgggtcacccccaagttcacccgaatggtggtcgctgcgctgttcggcgtgctggtcttgatgctcggcaacctcgtgctggcgatgttcaatgtcggcggcggtgaaggcttgggcttacgcagccccggaccgctggggatcatcttctcgctggtgtgcatcggcatcgcggcgttcagcttcctgatcgacttcgatgcggctgatcagatgattcgcgcgggagcaccggagaaggcggcatggggcgtcgcgttaggcctgaccgtaacgctggtctggttgtacatcgagatcctgcgcctgctcagttatctacagaatgagtag

SEQ ID NO: 4 - protein, 278 aa, Mycobacterium tuberculosis
VRETSNPVFRSLPKQRGGYAQFGTGTAQQGFPADPYLAPYREAKATRPLTIDDVVTKTGLTLAMLAGTAVVSYFLVASNVALAMPLTLVGALGGLALVLVATFGRKQDNPAIVLSYAALEGLFLGAISFVLANFTVASANAGVLIGEAILGTMGVFFGMLVVYKTGAIRVTPKFTRMVVAALFGVLVLMLGNLVLAMFNVGGGEGLGLRSPGPLGIIFSLVCIGIAAFSFLIDFDAADQMIRAGAPEKAAWGVALGLTVTLVWLYIEILRLLSYLQNE

SEQ ID NO: 5 - DNA, 357 bp, Mycobacterium tuberculosis
atgctgacgtgtgagatgcgggaatcggccctggctcgactcggccgggctctggctgatccgacgcggtgccggattctggtggcgttgctggatggcgtttgctatcccggccagctagctgcgcacctcgggttgacccgatcgaatgtgtccaaccatctgtcgtgtttgcggggctgcgggctggtagtcgcaacctatgagggccggcaggttcggtatgcgctggccgacagtcacctggcgcgagccttgggcgagttggtccaggtcgttctcgcggtggataccgaccaaccctgtgtcgccgagcgcgccgcgtccggggaggcggtcgagatgacaggtagctga

SEQ ID NO: 6 - protein, 118 aa, Mycobacterium tuberculosis
MLTCEMRESALARLGRALADPTRCRILVALLDGVCYPGQLAAHLGLTRSNVSNHLSCLRGCGLVVATYEGRQVRYALADSHLARALGELVQVVLAVDTDQPCVAERAASGEAVEMTGS

SEQ ID NO: 7 - DNA, 1248 bp, Mycobacterium tuberculosis
atgaccgtgccagcaccgtcgcagctcgacttgcgtcaagaggtgcacgacgccgcacgccgcgcccgggtggccgcccgccggctggcatcgctgccgacgactgtcaaagaccgcgcgctgcacgcggctgccgacgagctactggctcaccgcgaccagatcctggcggccaacgccgaagacctgaacgcggcgcgcgaggcggacaccccggccgccatgctggaccggttgtccttgaacccgcaacgagtcgacggtatcgccgccgggttgcggcaagtcgcgggactgcgcgatccggtcggtgaagtgctgcgtggctataccctgcccaacgggctgcagctgcgccagcagcgcgtccccctgggcgtggtcggcatgatctacgagggccgccccaatgtcaccgtggatgccttcgggctgacactcaagtcgggtaacgctgcattgctgcgcggcagctcgtcggccgcaaagtccaacgaggccctggtggcggtgttacgcaccgcgctggtcggcctggagctgccggccgacgcggtccagctgctgtcggctgccgaccgcgccaccgtcactcacctgattcaggcccgcggcctggtcgatgtggtgattccacgcgggggagcgggcctgatcgaggcggtcgtacgcgatgcccaggtgcccaccatcgagaccggcgtcgggaactgccatgtctacgtgcaccaagcggccgacctggacgtggccgagcgtatcttgctgaactccaagacgcggcggcccagcgtctgcaacgccgccgagacgctgctggtcgacgcagcgatcgccgaaacggcgttgcctcgattgctggccgccctgcagcacgccggtgtcaccgtacatctcgacccggacgaggccgacctgcgccgcgaatacctgtcgctggacatcgcggtggcggtggtcgacggtgtcgacgctgccatcgcccatatcaacgaatacggcaccgggcacacagaagcgattgtgaccaccaatcttgatgcggcccaacgctttaccgaacagatcgatgcggccgcggtgatggtgaacgcatcaacggcgttcaccgacggcgagcaattcggcttcggcgccgagatcggcatctccacccagaaactgcatgcccgcggaccgatgggactaccggaattgacgtcgaccaagtggatcgcatggggagccggccacacccgtccggcctga

SEQ ID NO: 8 - protein, 415 aa, Mycobacterium tuberculosis
MTVPAPSQLDLRQEVHDAARRARVAARRLASLPTTVKDRALHAAADELLAHRDQILAANAEDLNAAREADTPAAMLDRLSLNPQRVDGIAAGLRQVAGLRDPVGEVLRGYTLPNGLQLRQQRVPLGVVGMIYEGRPNVTVDAFGLTLKSGNAALLRGSSSAAKSNEALVAVLRTALVGLELPADAVQLLSAADRATVTHLIQARGLVDVVIPRGGAGLIEAVVRDAQVPTIETGVGNCHVYVHQAADLDVAERILLNSKTRRPSVCNAAETLLVDAAIAETALPRLLAALQHAGVTVHLDPDEADLRREYLSLDIAVAVVDGVDAAIAHINEYGTGHTEAIVTTNLDAAQRFTEQIDAAAVMVNASTAFTDGEQFGFGAEIGISTQKLHARGPMGLPELTSTKWIAWGAGHTRPA

SEQ ID NO: 9 - DNA, 1017 bp, Mycobacterium tuberculosis
atggtgcagctgtatgtctcggactccgtgtcgcggatcagctttgccgacggccgggtgatcgtgtggagcgaggagctcggcgagagccagtatccgatcgagacgctggacggcatcacgctgtttgggcggccgacgatgacaacgcccttcatcgttgagatgctcaagcgtgagcgcgacatccagctcttcacgaccgacggccactaccagggccggatctcaacacccgacgtgtcatacgcgccgcggctccgtcagcaagttcaccgcaccgacgatcctgcgttctgcctgtcgttaagcaagcggatcgtgtcgaggaagatcctgaatcagcaggccttgattcgggcacacacgtcggggcaagacgttgctgagagcatccgcacgatgaagcactcgctggcctgggtcgatcgatcgggctccctggcggagttgaacgggttcgagggaaatgccgcaaaggcatacttcaccgcgctggggcatctcgtcccgcaggagttcgcattccagggccgctcgactcggccgccgttggacgccttcaactcgatggtcagcctcggctattcgctgctgtacaagaacatcataggggcgatcgagcgtcacagcctgaacgcgtatatcggtttcctacaccaggattcacgagggcacgcaacgttggcgagcgacctcatggaggtatggcgggcgccgatcatcgatgacaccgtacttcgattgatcgcggacggtgtggtcgacacccgggctttcagcaagaactccgacacgggggccgtcttcgcgacacgggaagccacacgatccatcgcgcgcgcctttggtaatcgaatcgcacgaaccgccacctacatcaaaggcgatcctcaccgatacacctttcagtacgccctcgacttgcaactgcaaagcctcgtgcgtgttatcgaagccgggcacccgtcgcggctcgtcgatatcgatatcacctccgagccatccggagcctaa

SEQ ID NO: 10 - protein, 338 aa, Mycobacterium tuberculosis
MVQLYVSDSVSRISFADGRVIVWSEELGESQYPIETLDGITLFGRPTMTTPFIVEMLKRERDIQLFTTDGHYQGRISTPDVSYAPRLRQQVHRTDDPAFCLSLSKRIVSRKILNQQALIRAHTSGQDVAESIRTMKHSLAWVDRSGSLAELNGFEGNAAKAYFTALGHLVPQEFAFQGRSTRPPLDAFNSMVSLGYSLLYKNIIGAIERHSLNAYIGFLHQDSRGHATLASDLMEVWRAPIIDDTVLRLIADGVVDTRAFSKNSDTGAVFATREATRSIARAFGNRIARTATYIKGDPHRYTFQYALDLQLQSLVRVIEAGHPSRLVDIDITSEPSGA

SEQ ID NO: 11 - DNA, 363 bp, Mycobacterium tuberculosis
gtgtcgcagagtatgtacagctacccggcgatgacggccaatgtcggagacatggccggttatacgggcacgacgcagagcttgggggccgatatcgccagtgagcgcaccgcgccgtcgcgtgcttgccaaggtgatctcgggatgagtcatcaggactggcaggcccagtggaatcaggccatggaggctctcgcgcgggcctaccgtcggtgccggcgagcactacgccagatcggggtgctggaaaggccggtaggcgattcgtcagactgcggaacgattagggtggggtcgttccggggtcggtggctggacccgcgccatgcgggtccagccacggccgccgacgccggagactaa

SEQ ID NO: 12 - protein, 120 aa, Mycobacterium tuberculosis
VSQSMYSYPAMTANVGDMAGYTGTTQSLGADIASERTAPSRACQGDLGMSHQDWQAQWNQAMEALARAYRRCRRALRQIGVLERPVGDSSDCGTIRVGSFRGRWLDPRHAGPATAADAGD

SEQ ID NO: 13 - DNA, 1209 bp, Mycobacterium tuberculosis
atggcatcgggtagcggtctttgcaagacgacgagtaactttatttggggccagttactcttgcttggagagggaatccccgacccaggcgacattttcaacaccggttcgtcgctgttcaaacaaatcagcgacaaaatgggactcgccattccgggcaccaactggatcggccaagcggcggaagcttacctaaaccagaacatcgcgcaacaacttcgcgcacaggtgatgggcgatctcgacaaattaaccggcaacatgatctcgaatcaggccaaatacgtctccgatacgcgcgacgtcctgcgggccatgaagaagatgattgacggtgtctacaaggtttgtaagggcctcgaaaagattccgctgctcggccacttgtggtcgtgggagctcgcaatccctatgtccggcatcgcgatggccgttgtcggcggcgcattgctctatctaacgattatgacgctgatgaatgcgaccaacctgaggggaattctcggcaggctgatcgagatgttgacgaccttgccaaagttccccggcctgcccgggttgcccagcctgcccgacatcatcgacggcctctggccgccgaagttgcccgacattccgatccccggcctgcccgacatcccgggcctacccgacttcaaatggccgcccacccccggcagcccgttgttccccgacctcccgtcgttcccagggttccccgggttcccggagttccccgccatccccgggttccccgcactgcccgggttgcccagcattcccaacttgttccccggcttgccgggtctgggcgacctgctgcccggcgtaggcgatttgggcaagttacccacctggactgagctggccgctttgcctgacttcttgggcggcttcgccggcctgcccagcttgggttttggcaatctgctcagctttgccagtttgcccaccgtgggtcaggtgaccgccaccatgggtcagctgcaacagctcgtggcggccggcggtggccccagccaactggccagcatgggcagccaacaagcgcaactgatctcgtcgcaggcccagcaaggaggccagcagcacgccaccctcgtgagcgacaagaaggaagacgaggaaggcgtggccgaggcggagcgtgcacccatcgacgctggcaccgcggccagccaacgggggcaggaggggaccgtcctttga

SEQ ID NO: 14 - protein, 402 aa, Mycobacterium tuberculosis
MASGSGLCKTTSNFIWGQLLLLGEGIPDPGDIFNTGSSLFKQISDKMGLAIPGTNWIGQAAEAYLNQNIAQQLRAQVMGDLDKLTGNMISNQAKYVSDTRDVLRAMKKMIDGVYKVCKGLEKIPLLGHLWSWELAIPMSGIAMAVVGGALLYLTIMTLMNATNLRGILGRLIEMLTTLPKFPGLPGLPSLPDIIDGLWPPKLPDIPIPGLPDIPGLPDFKWPPTPGSPLFPDLPSFPGFPGFPEFPAIPGFPALPGLPSIPNLFPGLPGLGDLLPGVGDLGKLPTWTELAALPDFLGGFAGLPSLGFGNLLSFASLPTVGQVTATMGQLQQLVAAGGGPSQLASMGSQQAQLISSQAQQGGQQHATLVSDKKEDEEGVAEAERAPIDAGTAASQRGQEGTVL